(19) United States
(12) Patent Application Publication
(10) Pub. No.: US 2008/0051462 A1
(43) Pub. Date: Feb. 28, 2008

(54) Methods for treating genetically-defined proliferative disorders with HSP90 inhibitors

(75) Inventors: Lawrence C. Fritz, San Diego, CA (US); Francis J. Burrows, San Diego, CA (US)

Correspondence Address: BIOGEN IDEC / FINNEGAN HENDERSON, LLP, 901 NEW YORK AVENUE, NW, WASHINGTON, DC, 20001-4413, US

(73) Assignee: Conforma Therapeutics Corp.

(21) Appl. No.: 11/779,243

(22) Filed: Jul. 17, 2007

Related U.S. Application Data
(63) Continuation of application No. 10/469,469, filed on Aug. 27, 2003, now abandoned.
(60) Provisional application No. 60/272,751, filed on Mar. 1, 2001.

(51) Int. Cl. A61K 31/135; A61P 43/00; C12Q 1/68

(52) U.S. Cl. 514/646; 435/6

(57) ABSTRACT

The invention relates generally to methods of treating cell proliferative diseases with HSP90 inhibitors and, depending on the specific aspect and embodiment(s) claimed, to the treatment of proliferative diseases that are associated with fusion proteins, e.g., berab1, or mutant proteins or cellular protein isoforms, e.g., mutant forms of p53.

### FIELD OF THE INVENTION

The field of the invention relates to chemotherapeutic treatments of proliferative disorders, including rheumatoid arthritis and neoplasias.

### BACKGROUND OF THE INVENTION

The following description includes information that may be useful in understanding the present invention. It is not an admission that any of the information provided herein is prior art, or relevant, to the presently claimed inventions, or that any publication specifically or implicitly referenced is prior art.

The eukaryotic heat shock protein 90s (HSP90s) are ubiquitous chaperone proteins that are involved in folding, activation and assembly of a wide range of proteins, including key proteins involved in signal transduction, cell cycle control and transcriptional regulation. HSP90 proteins are highly conserved in nature (see, e.g., NCBI accession # P07900 (SEQ ID NO: 318) and XM 004515 (SEQ ID NOs: 319 and 320) (human α and β HSP90, respectively), P11499 (SEQ ID NO: 321) (mouse), AAB23369 (SEQ ID NO: 322) (rat), P46633 (SEQ ID NO: 323) (chinese hamster), JC1468 (SEQ ID NO: 324) (chicken), AAF69019 (SEQ ID NO: 325) (fleshfly), AAC21566 (SEQ ID NO: 326) (zebrafish), AAD30275 (SEQ ID NO: 327) (salmon), AAC48718 (SEQ ID NO: 328) (pig), NP 015084 (SEQ ID NO: 329) (yeast), and CAC29071 (SEQ ID NO: 330) (frog).

Researchers have reported that HSP90 chaperone proteins are associated with important signaling proteins, such as steroid hormone receptors and protein kinases, including many that are implicated in tumorigenesis, e.g., Raf-1, EGFR, v-Src family kinases, Cdk4, and ErbB-2 (Buchner J., 1999, TIBS, 24:136-141; Stepanova, L. et al., 1996, Genes Dev. 10:1491-502; Dai, K. et al., 1996, J. Biol. Chem. 271:22030-4). In vivo and in vitro studies indicate that certain co-chaperones, e.g., Hsp70, p60/Hop/Sti1, Hip, Bag1, HSP40/Hdj2/Hsj1, immunophilins, p23, and p50, may assist HSP90 in its function (Caplan, A., 1999, Trends in Cell Biol., 9: 262-68).

Ansamycins are antibiotics derived from Streptomyces hygroscopicus which are known to inhibit HSP90s. These antibiotics, e.g., herbimycin A (HA) and geldanamycin (GM), as well as other HSP90 inhibitors such as radicicol, bind tightly to an N-terminal pocket in HSP90 (Stebbins, C. et al., 1997, Cell, 89:239-250). This pocket is highly conserved and has weak homology to the ATP-binding site of DNA gyrase (Stebbins, C. et al., supra; Grenert, J. P. et al., 1997, J. Biol. Chem., 272:23843-50). ATP and ADP have been shown to bind this pocket with low affinity, and HSP90 itself has been shown to have weak ATPase activity (Proromou, C. et al., 1997, Cell, 90: 65-75; Panaretou, B. et al., 1998, EMBO J, 17: 4829-36). In vitro and in vivo studies have demonstrated that occupancy of the N-terminal pocket of HSP90 by ansamycins and other inhibitors alters HSP90 function and inhibits client protein folding. At high concentrations, ansamycins and other HSP90 inhibitors have been shown to prevent binding of client protein substrates to HSP90 (Scheibel, T., H. et al., 1999, Proc. Natl. Acad. Sci. USA 96:1297-302; Schulte, T. W. et al., 1995, J. Biol. Chem. 270:24585-8; Whitesell, L., et al., 1994, Proc. Natl. Acad. Sci. USA 91:8324-8328). Ansamycins have also been demonstrated to inhibit the ATP-dependent release of chaperone-associated protein substrates (Schneider, C., L. et al., 1996, Proc. Natl. Acad. Sci. USA, 93:14536-41; Sepp-Lorenzino et al., 1995, J. Biol. Chem. 270:16580-16587), and some of these substrates have been shown to be degraded by a ubiquitin-dependent process in the proteasome (Schneider, C., L., supra; Sepp-Lorenzino, L., et al., 1995, J. Biol. Chem., 270:16580-16587; Whitesell, L. et al., 1994, Proc. Natl. Acad. Sci. USA, 91: 8324-8328).

This substrate destabilization occurs in tumor and nontransformed cells alike and has been shown to be especially effective on a subset of signaling regulators, e.g., Raf (Schulte, T. W. et al., 1997, Biochem. Biophys. Res. Commun. 239:655-9; Schulte, T. W., et al., 1995, J. Biol. Chem. 270:24585-8), nuclear steroid receptors (Segnitz, B., and U. Gehring. 1997, J. Biol. Chem. 272:18694-18701; Smith, D. F. et al., 1995, Mol. Cell. Biol. 15:6804-12), v-src (Whitesell, L., et al., 1994, Proc. Natl. Acad. Sci. USA 91:8324-8328) and certain transmembrane tyrosine kinases (Sepp-Lorenzino, L. et al., 1995, J. Biol. Chem. 270:16580-16587) such as EGF receptor (EGFR) and Her2/Neu (Hartmann, F., et al., 1997, Int. J. Cancer 70:221-9; Miller, P. et al., 1994, Cancer Res. 54:2724-2730; Mimnaugh, E. G., et al., 1996, J. Biol. Chem. 271:22796-801; Schnur, R. et al., 1995, J. Med. Chem. 38:3806-3812). The ansamycin-induced loss of these proteins leads to the selective disruption of certain regulatory pathways and results in growth arrest at specific phases of the cell cycle (Muise-Heimericks, R. C. et al., 1998, J. Biol. Chem. 273:29864-72), and apoptsosis of cells so treated (Vasilevskaya, A. et al., 1999, Cancer Res., 59:3935-40).

Growth arrest of this sort, provided it can be made selective, has important ramifications for the treatment of certain proliferative disorders, including cancer. Whereas cancer treatments have thus far been limited to traditional surgical removal, radiation, and/or chemotherapy, and whereas these procedures have been more or less successful, a need remains to develop additional therapies with increased efficacy and decreased side-effects that can be used alone or in combination with existing therapies. There particularly remains a need for cancer treatments that target specific cancer types. The present invention satisfies these needs and provides related advantages as well.

### SUMMARY OF THE INVENTION

Applicants report that many proliferative disorders are associated with aberrant proteins that exhibit a dependence on HSP90. In some cases this dependence manifests as a heightened sensitivity to HSP90 inhibitors such that affected cells can be selectively treated using a dosage that is effective against the aberrant cells but which is ineffective or less effective against normal cells. The aberrant proteins may also exhibit increased proteosome-dependent degradation when in the presence of HSP90 inhibitors. While the invention is not limited by mechanism, increased dependence, sensitivity, and/or disposition to preferential degradation may advantageously be used to treat corresponding proliferative diseases according to the methods of the invention.

Among others, the invention targets two groups of aberrant proteins in particular and the corresponding proliferative disorders they are associated with. Within the first group are fusion proteins generated as a result of non-random chromosomal aberrations (such as translocations, deletions and inversions) that juxtapose parts of the coding sequences of two normal cellular proteins (Rabbitts, T., 1994, Nature 372:143-149). Duplication of genetic material within a chromosome resulting in a augmented or semi-duplicative transcripts is also a possibility. Within the second group are mutants and isoforms of cellular proteins that override, dominate, or otherwise obscure the natural gene products and their function. For example, mutants and isoforms of p53 family proteins and other tumor suppressor gene products can act as dominant-negative inhibitors of the corresponding normal protein in heterozygous tumor cells (Blagosklonny, M., et al, 1995, Oncogene, 11:933-939. Other examples include virally-encoded species of certain kinases, such as v-src and other dominantly-acting mutant oncogene products (Uehara, Y. et al., 1985, supra).

Accordingly, in a first aspect the invention features a method of treating a patient having a genetically-defined proliferative disease characterized by a non-random chromosomal aberration. This aberration produces or is capable of producing an oncogenic fusion protein. The method in its broadest embodiment includes (a) providing a cell, tissue, or fluid sample of a patient suspected of having a genetically-defined proliferative disease; (b) identifying in the cell, tissue, or fluid sample one or more characteristics indicative of the proliferative disease; and (c) administering to the patient a pharmaceutically effective amount of an HSP90-inhibiting compound.

The patient may be any organism that can manifest a proliferative disease characterized by an oncogenic fusion protein, which disease is responsive to HSP90 inhibitors. Preferably, but not necessarily, the organism is an animal, more preferably a mammal, and most preferably a human.

In preferred embodiments, the inhibitory compound is an ansamycin including but not limited to, e.g., geldanamycin, the geldanamycin derivative, 17-AAG, herbimycin A, and/or macbecin. Most preferably, the ansamycin is 17-AAG. These and other ansamycins and methods of preparing them are well-known in the art. See, e.g., U.S. Pat. Nos. 3,595,955, 4,261,989, 5,387,584, and 5,932,566. Although preferably the compound is an ansamycin, the method may make use of any compound, synthetic or nonsynthetic, that can inhibit HSP90. Preferably, the inhibitor binds the ATP-binding site of HSP90, or an HSP90 homolog. Radicicol is a nonsynthetic example of a compound useful in the invention described and claimed herein. Libraries of small molecules, synthetic and/or nonsynthetic exist or can be made according to routine, well-known methods and screened for HSP90 binding and/or inhibitory activity. These molecules with HSP90 binding and/or inhibitory activity are also useful in the methods of the invention.

In the identifying step of the invention, which is carried out prior to diagnosis where/when there is no previous diagnosis, any technique can be used that can identify or predict a proliferative disorder targetable by HSP90 inhibitors. Especially preferred are antibody-based and nucleic acid hybridization and/or amplification techniques. Immunoprecipitation, western blotting, and immunoblotting are illustrative examples of antibody-based methods. The antibodies may be monoclonal and/or polyclonal. Illustrative examples of nucleic acid hybridization-based techniques involve Southern blotting, northern blotting, and dot-blotting. Illustrative examples of nucleic acid amplification include standard polymerase chain reactions and variations thereof, e.g., reverse transcriptase-PCR(RT-PCR). The latter is especially useful for identifying levels of gene expression. Other techniques such as the ligase chain reaction (LCR) are also well-known and have the ability to distinguish an aberrant gene (and indirectly a protein product produced therefrom) from a normal one, or at least predict genotype and/or phenotype. Other methods of identification include ligand-binding assays and gel-retardation assays that display characteristic binding affinities and/or mobility profiles for normal and variant proteins. Where the fusion protein is also an enzyme, one can establish and/or measure aberrance by enzymatic activity (or lack thereof). Conventional and derivative karyotyping and cytochemical techniques can also be used to identify a proliferative disorder of the invention prior to administration of HSP90-inhibitors. One such method is fluorescent in situ hybridization (FISH).

In some embodiments, the proliferative disease is a hematopoietic disorder including but not limited to one selected from the group consisting of T or B cell lymphomas, chronic myeloid leukemias (CMLs), acute promyelocytic leukemias (APLs), acute lymphoid or lymphoblastic leukemias (ALLs), acute myeloid leukemias (AMLs), non-Hodgkin lymphomas (NHLs), and chronic myelomonocytic leukemias (CMMLs). In other embodiments, the disease is characterized by a solid tumor, preferably including but not limited to papillary thyroid carcinoma, Ewing's sarcoma, melanoma, liposarcoma, rhabdomyosarcoma, synovial sarcoma. The embodiments are not necessarily mutually exclusive of one another, and treatment of multiple distinct diseases may simultaneously be effected in a given patient, as the invention has broad-spectrum merit against a variety of different proliferative disorders.

Targeted fusion proteins may contain one or more functional domains or portions thereof, e.g., kinases, DNA binding motifs, etc. Such domains are well-known in the art. FIG. 1 illustrates several types of these domains, and the specific fusion proteins, genes, and diseases they can be associated with.

Administration may be by a variety of means. In some preferred embodiments, administration is made ex vivo, e.g., removing and treating blood or tissue that is thereafter administered back into the patient. Alternatively, or in combination, administration may be intralesional, e.g., administered to the site of a solid tumor, and/or parenteral. These constitute just some of the many different modes of administration that can be used. Others are described herein.

In other embodiments, the HSP90-inhibiting compound has an IC₅₀ that is higher (preferably two-fold, more preferably five-fold, and most preferably ten-fold) for cells that do not have characteristics indicative of the proliferative disorder as compared with those cells that do have such characteristics.

In other embodiments, the patient may be tested pre- and/or post-administration for sensitivity and or effect of one or more HSP90 inhibitors. This may be done in vitro or in vivo.

Numerous non-random chromosomal aberrations exist that are associated with proliferative disorders. These include but are not limited to chromosomal translocations, inversions, and deletions. Duplications also account for some aberrant chromosomes and aberrant resulting gene products. All aberrations can be targeted in various aspects of the invention. Illustrative examples of specific aberrations include those listed in FIG. 1, which is adapted from Table 1 of Rabbitts, Nature 372:143-149 (1994), and others including but not limited to: inv14 (q11; q32), t(9; 22)(q34; q11), t(1; 19)(q23; p13.3), t(17; 19)(q22; p13), t(15; 17)(q21-q11-22), t(11; 17)(q23; q21.1), t(4; 11)(q21; q23), t(9; 11)(q21; q23), t(11; 19)(q23; p13), t(X; 11)(q13; q23), t(1; 11)(p32; q23), t(6; 11)(q27; q23), t(11; 17)(q23; q21), t(8; 21)(q22; q22), t(3; 21)(q26; q22), 5(16; 21)(p11; q22), t(6; 9)(p23; q34), 9; 9?, t(4; 16)(q26; p 13), inv(2; 2)(p13; p 11.2-14), inv(16)(p13q22), t(5; 12)(q33; p13), t(2; 5)(2p23; q35), t(9:12)(q34:p13), del(12p), t(9;22), +8, +Ph,i(17q), t(15;17)(q22;q12), t(11;17)(q23;q12), t(16:16)(p13;q22), inv(16)(p13;q22), t(9;11)(p22;q23), t(1;22)(p13;q13), t(3;3)(q21;q26), inv(3)(q21q26), t(3;5)(q21;q31), t(3;5)(q25;q34), t(7;11)(p15;p15), t(8;16)(p11;p13), t(9;12)(q34;p13), t(12;22)(p13;q13), del(5q), del(7q), del(20q), t(11q23), t(12;21)(p13;q22), t(5;12)(q31;p13), t(1;12)(q25;p13), t(12;15)(p13;q25), t(1;12)(q21;p13), t(12;21)(q13;p32), and t(5;7)(q33;q11.2). These are merely a sampling of the many chromosomal aberrations well-known in the art that give rise to particular proliferative disorders treatable according to the invention. For these and others, see, e.g., the National Center for Biotechnology Information (NCBI) databases, including, e.g., the Online Mendelian Inheritance in Man (OMIM) database and related links to nucleotide and protein sequences. For purposes of the present invention, the underlying genetic sequences affected are for the most part known and/or may be deduced using techniques routine in the art.

Targeted in particularly preferred embodiments of the invention are chromosomal aberrations corresponding to t(9; 22)(q34; q11) that give rise to bcr-abl fusion proteins, chronic myelogenous leukemia (CML) and, in some cases, acute lymphoid or lymphoblastic leukemia (for ALL, see, e.g., Erikson et al., Heterogeneity of chromosome 22 breakpoint in Philadelphia-positive (Ph+) acute lymphocytic leukemia, Proc. Nat. Acad. Sci. 83: 1807-1811 (1986))).

In a second aspect, the invention features a method of treating cancerous cells in a heterogeneous population of cells. The heterogeneous population includes both cancerous and noncancerous cells, and the cancerous cells are further characterized by fusion proteins that are not produced in the noncancerous cells. The method includes administering to the heterogeneous population a pharmaceutically effective amount of an HSP90-inhibiting compound. The population may be tested by separation of samples from each population into separate subpopulations, cancerous or noncancerous, e.g., where cultured cells of each are tested in parallel for response and/or susceptibility to an HSP90-inhibitor or candidate inhibitor molecule. Alternatively, the population may be mixed, e.g., in an ex vivo procedure in which cells of a patient, e.g., blood, are treated and administered back to the patient or to another individual. This method otherwise tracks the various described and/or claimed embodiments and/or combinations of embodiments of the first aspect.

In a third aspect, the invention features a method of treating a patient having a proliferative disease associated with a mutant protein or cellular protein isoform dependent on HSP90, or which disease is otherwise sensitive to HSP90 inhibitors. The method includes (a) providing a cell, tissue, or fluid sample of a patient suspected of having said proliferative disease; (b) identifying in the cell, tissue, or fluid sample one or more characteristics indicative of a mutant or cellular protein isoform; and (c) administering to the patient a pharmaceutically effective amount of an HSP90-inhibiting compound.

In preferred embodiments, the mutant protein or cellular protein isoform is selected from the group consisting of src, RET, p53, p51, p63, and p73. Most preferably selected are isoformis of p53 selected from N239S, C176R, and R213*, Y236delta, C174Y, M133T, G245D, E258K, 1-293delta, G245C, R248W, E258K, R282W, R175H, R280K, V143A, R175H, P177S, H178P, H179R, R181P, 238-9delta, G245S, G245D, M246R, R248Q, R249S, R273H, R273c, R273L, and D281Y.

In another preferred embodiment, the proliferative disease to be treated is rheumatoid arthritis.

In some embodiments, the mutant protein or cellular protein isoform may give rise to a dominant negative phenotype In other embodiments, the mutant or cellular protein isoform may give rise to a dominant positive mutant. In either embodiment, the patient may be heterozygous for the normal cellular gene. Other embodiments track those listed for the preceding aspects.

In a fourth aspect, the invention features a method of selectively treating cells that express a mutant protein or cellular protein isoform associated with a proliferative disorder and which mutant/isoform is dependent on HSP90, or which disease is otherwise sensitive to HSP90 inhibitors. The method includes (a) providing a population of cells in which at least some of the population express a mutant protein or cellular protein isoform that is dependent on HSP90 or which are otherwise sensitive to HSP90 inhibitors. The method further includes administering to the population a pharmaceutically effective amount of an HSP90-inhibiting compound. The embodiments for this aspect may otherwise track preceding embodiments.

The foregoing aspects contemplate treatment of existing cell proliferative disorders. It is expected that the invention may also find use in prophylactic prevention of various proliferative disorders of the invention. Further, and where appropriate, each of the embodiments discussed above and different combinations thereof, including subgenus and sub-Markush groups, may cross-apply to each of the different aspects of the invention. Further, where sequence listings are provided, the invention may in some aspects contemplate subsequences of the primary sequence listings. Any subsequence within such primary listing is also contemplated for the invention, as well as all allelic variants, and mutant variants and isoforms thereof, as well as corresponding homologs from other organisms and species. Sequences contiguous with and/or in addition to the listed sequences and their above equivalents are also contemplated.

Advantages of the invention include broad-acting treatment or prophylaxis directed to a variety of different proliferative disorders. Other advantages include the efficient and rapid diagnosis and care of patients suffering from proliferative disorders, with minimal apparent adverse effects. Still other advantages, aspects, and embodiments will be apparent from the figures, the detailed description, and the claims.

### BRIEF DESCRIPTION OF THE DRAWINGS

FIG. 1 illustrates various genetically defined diseases characterized by non-random chromosomal aberrations that give rise to oncogenic fusion proteins. These illustrative aberrations, diseases, and fusion proteins are targeted in various embodiments of the invention. Other targeted aberrations, diseases, and fusion proteins may be found in the specification and in sources commonly known in the art, e.g., the NCBI and GenBank databases, and journal literature.

### DETAILED DESCRIPTION OF THE INVENTION

### Definitions

As used herein and in the claims the following terms have the following meanings:

A “genetically-defined disease” is one with a basis in DNA. Genetically defined diseases of the invention include “cell proliferative disorders” wherein unwanted cell proliferation of one or more subset(s) of cells in a multicellular organism occurs, resulting in harm, for example, pain or decreased life expectancy to the organism. “Cell proliferative disorders” refer to disorders wherein unwanted cell proliferation of one or more subset(s) of cells in a multicellular organism occurs, resulting in harm, for example, pain or decreased life expectancy to the organism. Cell proliferative disorders include, but are not limited to, cancers, tumors, benign tumors, blood vessel proliferative disorders, autoimmune disorders and fibrotic disorders. These disorders are not necessarily independent. For example, fibrotic disorders may be related to, or overlap with, blood vessel disorders, e.g., atherosclerosis (which is characterized herein as a blood vessel disorder that is associated with the abnormal formation of fibrous tissue).

A “non-random chromosomal aberration” is one that occurs with a nonrandom frequency or is selected for in a population of individuals. Chromosomal aberrations of the invention include translocations, i.e., relocation of a fragment of one chromosome onto another chromosome; inversions, i.e., wherein pieces of a chromosome rotate within the same chromosome, and deletions, i.e., wherein fragments of a chromosome are lost thereby juxtaposing pieces of DNA that previously did not reside immediately beside each other.

An “oncogenic fusion protein” is a protein that is non-natural in and of itself but that may contain one or more pieces of other proteins that may or may not naturally occur within a cell. The fusion protein functions by improperly stimulating cell growth, directly or indirectly. In the context of the invention, the term is also associated with a cellular proliferative disease and is preferably encoded by a nucleic acid found in the cell, e.g., as part of a non-random chromosomal aberration. An oncogenic fusion protein may contain domains or portions thereof, e.g., kinases and/or DNA binding proteins that are well known in the art, or else predicted from their structure to behave as such.

A “fusion” may relate to, as appropriate to a given context, a fusion chromosome, an abnormal mRNA transcribed from the fused portion of the chromosome, or a polypeptide product translated from the abnormal mRNA that is transcribed from the fusion chromosome. These fusions may result from chromosomal deletions, insertions, and/or translocations. Domains or portions of different genes and gene products are frequently, although not necessarily always, brought together as a consequence of the fusion event. For example, an intragenic deletion can result in an intragenic fusion and give rise to an abnormal protein lacking a component from a second gene. More frequently it occurs that two genes or portions thereof are juxtaposed more or less, transcribed together as a single transcript, and translated together as a fusion protein bearing contributions from multiple genes or other chromosomal DNA pieces. In such fusions, reading frames can be preserved, e.g., as in preserved functional domains or portions thereof coming from two or more different genes, or else the reading frame can be disrupted, e.g., as in the case of a “missense” or “nonsense” event as these terms are known in the art.

By “providing a cell, tissue, or fluid sample of a patient suspected of having said genetically-defined disease” and “identifying one or more characteristics indicative of said disease in or on said cell, tissue, or fluid sample” can mean, although is not limited to the situation where, the sample is withdrawn from the patient in order to perform the analysis or analyses. Many invasive and noninvasive procedures exist, e.g., NMR, ultrasound and other imaging techniques, that can be used to diagnose, at least in part, an illness and its cause. For example, “tagged” antibodies or other ligands with affinity for a fusion protein or chromosomal aberrancy or aberrancy product of the invention can be used to make the diagnosis and/or assist in treatment according to methods of the invention.

“Characteristics indicative of said disease” may embrace phenotypes or genotypes and may be measured qualitatively or quantitatively by a variety of techniques. The characteristics may be observed with the naked eye or else through the assistance of a machine or other diagnostic technique(s). Exemplary techniques of measurement include but are not limited to immunoreactivity and/or precipitation, PCR, LCR, karyotyping, and fluorescence activated cell sorting (“FACS)” as those terms are known and understood in the art.

“Administering” can be by direct means, e.g., intralesional or by parenteral or peripheral administration to a patient, or else by indirect means, e.g., as by withdrawing a patient's cells, treating them, and then re-introducing them back into the patient. The latter constitutes an “ex vivo” technique.

An “HSP90-inhibiting compound” is one that disrupts the expression, structure, and/or function of an HSP90 chaperone protein and/or a protein that is dependent on HSP90. HSP90 proteins are highly conserved in nature (see, e.g., NCBI accession #'s P07900 and XM 004515 (human α and β HSP90, respectively), P11499 (mouse), AAB2369 (rat), P46633 (chinese hamster), JC1468 (chicken), AAF69019 (flesh fly), AAC21566 (zebrafish), AAD30275 (salmon), 002075 (pig), NP 015084 (yeast), and CAC29071 (frog). There are thus many different HSP90s, all with anticipated similar effect and similar inhibition capabilities. The HSP90 inhibitor used in the methods of the invention may be specifically directed against an HSP90 of the specific host patient or may be identified based on reactivity against an HSP90 homolog from a different species, or an artificial HSP90 variant. The inhibitors used may be ring-structured antibiotics, e.g., benzoquinone ansamycins, or other types of molecules, e.g., antisense nucleic acids and molecules such as radicicol.

An “ansamycin” includes but is not limited to geldanamycin, 17-AAG, herbimycin A, and macbecin. The specific ansamycin 17-AAG stands for 17-allylamino-17-demethoxygeldanamycin. This and other ansamycins that can be used are well-known in the art. See, e.g., U.S. Pat. Nos. 3,595,955, 4, 261, 989, 5,387,584, and 5,932,566. Ansamycins may be synthetic, naturally-occurring, or else derivatives of naturally occurring ansamycins that are prepared using standard chemical derivatization techniques.

A “pharmaceutically effective amount” means an amount which is capable of providing a therapeutic or prophylactic effect. The specific dose of compound administered according to this invention to obtain therapeutic and/or prophylactic effects will, of course, be determined by the particular circumstances surrounding the case, including, for example, the specific compound administered, the route of administration, the condition being treated, the individual being treated, and the tissue or cell type targeted (or not targeted). A typical daily dose (administered in single or divided doses) will contain a dosage level of from about 0.01 mg/kg to about 100 and more preferably 50 mg/kg of body weight of an active compound of this invention. Preferred daily doses generally will be from about 0.05 mg/kg to about 20 mg/kg and ideally from about 0.1 mg/kg to about 10 mg/kg.

A preferred therapeutic effect is the inhibition to some extent of the growth of cells causing or contributing to a cell proliferative disorder. A therapeutic effect will also normally, but need not, relieve to some extent one or more of the symptoms of a cell proliferative disorder other than cell growth or size of cell mass. In reference to the treatment of a cancer, a therapeutic effect refers to one or more of the following: 1) reduction in the number of cancer cells; 2) reduction in tumor size; 3) inhibition (i.e., slowing to some extent, preferably stopping) of cancer cell infiltration into peripheral organs; 3) inhibition (i.e., slowing to some extent, preferably stopping) of tumor metastasis; 4) inhibition, to some extent, of tumor growth; and/or 5) relieving to some extent one or more of the symptoms associated with the disorder.

In reference to the treatment of a cell proliferative disorder other than a cancer, a therapeutic effect refers to either: 1) the inhibition, to some extent, of the growth of cells causing the disorder; 2) the inhibition, to some extent, of the production of factors (e.g., growth factors) causing the disorder; and/or 3) relieving to some extent one or more of the symptoms associated with the disorder.

With respect to viral infections, the preferred therapeutic effect is the inhibition of a viral infection. More preferably, the therapeutic effect is the destruction of cells which contain the virus.

A “cancer” refers to one or more various types of benign or malignant neoplasms. In the case of the latter, these may invade surrounding tissues and may metastasize to different sites, as defined in Stedman's Medical Dictionary 25th edition (Hensyl ed. 1990).

The term “IC₅₀” is defined as the concentration of an HSP90 inhibitor required to achieve killing or other growth inhibition of 50% of the cells of a homogenous cell type population, or of a particular cell type, e.g., cancerous versus noncancerous, over a period of time. The IC₅₀ is preferably, although not necessarily, greater for normal cells than for cells exhibiting a proliferative disorder.

The term “mutant or isoform cellular protein” refers to a variation of a wild-type protein that occurs in a cell and has a particular function. The mutant or isoform cellular protein of the invention preferably associates with or gives rise to a proliferative disorder, e.g., a cancer, whereas the wild-type protein ordinarily does not.

General

As described and claimed herein, ansamycins and other HSP90 inhibitors can be used to treat two important classes of tumor-promoting (oncogenic) human proteins.

1. Oncogenic Fusion Proteins

The first class of target proteins of the invention are fusion proteins generated as a result of non-random chromosomal aberrations (such as translocations, deletions and inversions) that juxtapose parts of the coding sequences of two normal cellular proteins (Rabbitts, T., 1994, Nature 372:143-149) leading to the lineage-specific expression of a mutant fusion protein that has biological activities derived from both parent proteins (Barr, F, 1998, Nat. Genet. 19:121-124). Without being limiting of the invention, Applicants have discovered that these fusion proteins have a heightened dependence on HSP90 chaperone activity, and/or decreased stability in the presence of HSP90 inhibitors, thus making them selective targets for treatment with HSP90 inhibitors.

a. Bcr-abl as an Example

One example of heightened HSP90 dependence and inhibitor sensitivity is observed when chronic myelogenous leukemia (CML) cells harboring the fusion oncoprotein p210-bcr-abl are treated with HSP90 inhibitors. This fusion protein is degraded faster and more completely than wild type c-abl protein (An, W et al, 2000, Cell Growth and Differentiation 11: 355-360). Further experimental evidence that bcr-abl expressing leukemia cells are more sensitive to HSP90 inhibitors than are closely related bcr-abl-negative leukemia lines is found in Honma, Y et al, 1995, Int. J. Cancer 60:685-688, where it is reported that the IC₅₀ of herbimycin A in six bcr-abl expressing leukemia cell lines averaged 29.3 nM as compared to a mean IC₅₀ of 399.3 nM in a panel of four bcr-abl-negative leukemia lines. Illustrative protein and nucleic acid sequences corresponding to embodiments of bcr-abl fusions of the invention include but are not limited to those found in SEQ ID NOs 1-26 and subsequences thereof, which are further discussed below, along with corresponding NCBI accession numbers.

The normal Bcr gene occupies a region of about 135 kb on chromosome 22. It is expressed as mRNAs of 4.5- and 6.7-kb, which apparently encode for the same cytoplasmic 160-kD protein, and contains 23 exons as well as an unusual inverted repeat flanking the first exon. The BCR protein reportedly contains a unique serine/threonine kinase activity and at least two SH2 binding sites encoded in its first exon and a C-terminal domain that functions as a GTPase activating protein for p21 (rac) (Diekmann et al., Nature 351: 400-402 (1991). Chissoe et al., Genomics 27: 67-82 (1995), sequenced the complete BCR gene and greater than 80% of the human ABL gene, which are both involved in the t(9;22) translocation (Philadelphia chromosome) associated with more than 90% of chronic myelogenous leukemia, 25 to 30% of adult and 2 to 10% of childhood acute lymphoblastic leukemia, and rare cases of acute myelogenous leukemia. Comparison of the gene with its cDNA sequence revealed the positions of 23 BCR exons and putative alternative BCR first and second exons. From the sequence of four newly studied Philadelphia chromosome translocations and a review of several other previously sequenced breakpoints, Chissoe et al. found a variety of breakpoints and recombinations sites possible within the genes. Thus, despite the normal chromosomes and genes each being known (9 and 12; bcr and abl), and the fact that combinations of these genes are known to lead to forms of CML and ALL, the precise genetic breakpoint/recombination junctions that lead to these diseases can vary.

This heterogeneity likely also applies to some non bcr-abl chromosomal aberrations of the invention as well. Nevertheless, because the genes and/or chromosomes involved are known to have a part in the disorders, the disorders are said to be “genetically defined.”

B. Other Oncogenic Fusion Proteins

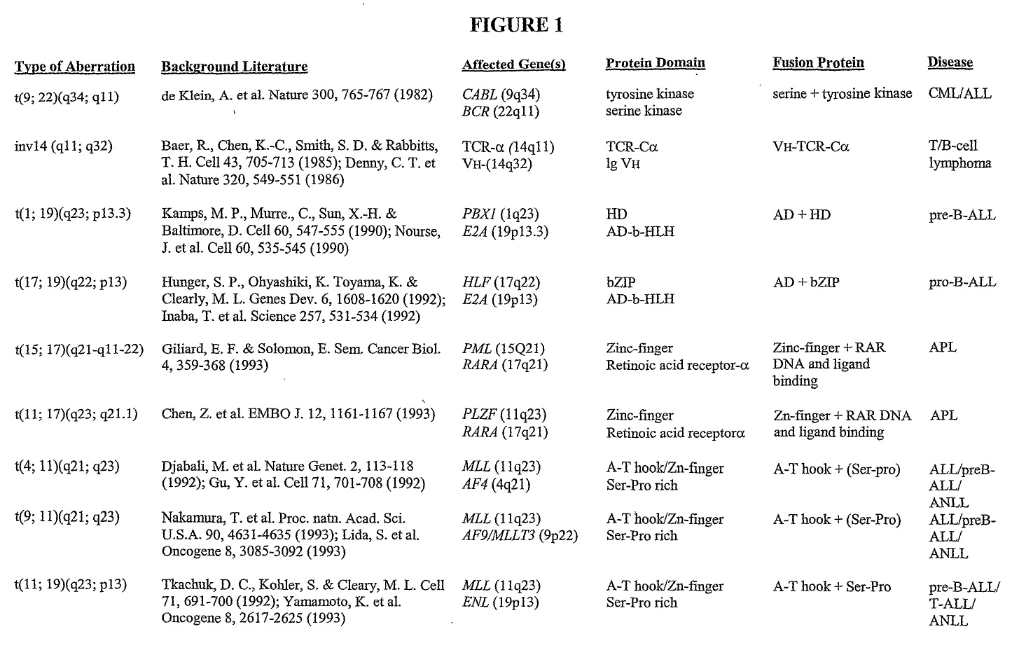

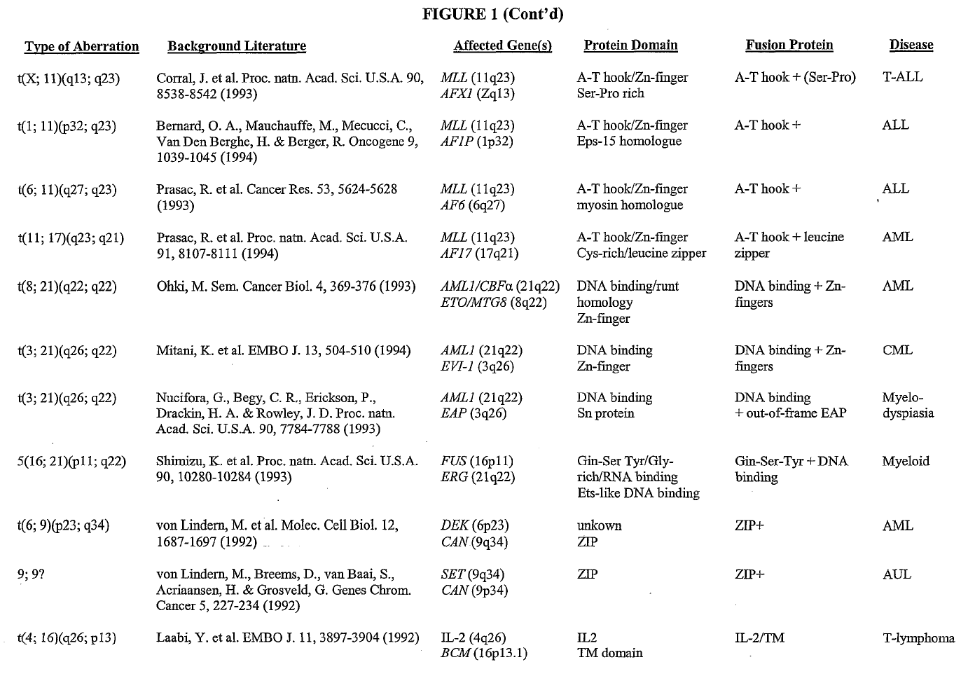

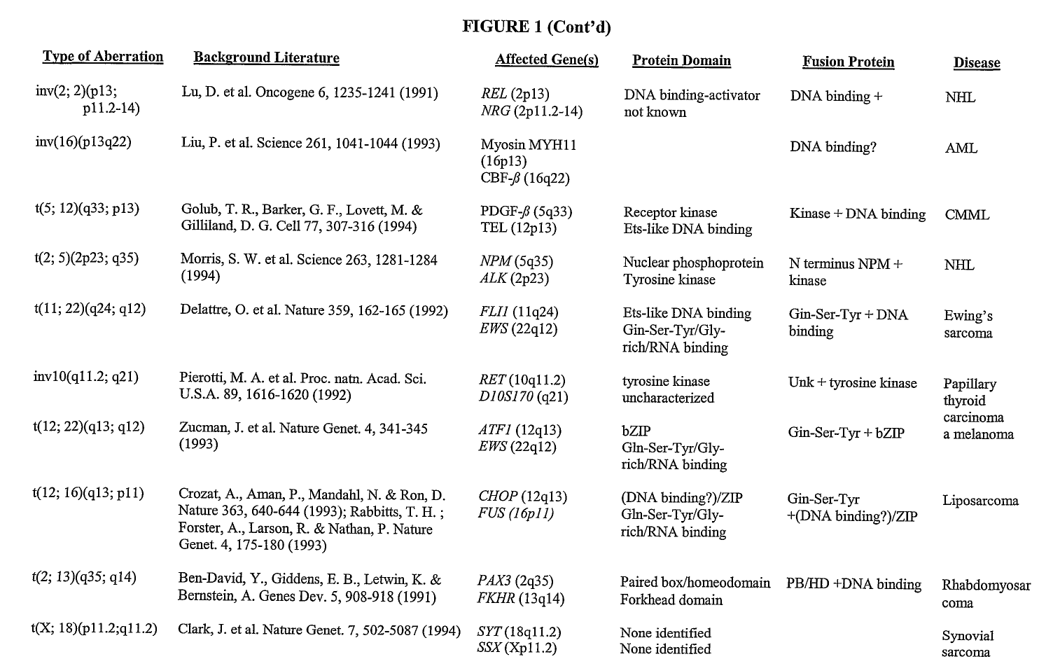

Oncogenic fusion proteins in general are thought to be inherently unstable. To the extent these unstable oncogenic fusion proteins make use of HSP90, they are susceptible of the methods claimed herein. Because the fusion genes and their protein products exert overtly oncogenic activity (Deininger, M et al, 2000, Cancer Res. 60:2049-2055), preferential degradation of these labile proteins induced by HSP90 inhibitors will have therapeutic value in diseases where the fusion protein is expressed. The present invention thus includes treatment of patients with tumors that are dependent upon other oncogenic fusion proteins that arise from non-random genetic aberrations. An illustrative but nonexhaustive list of these tumors is included in FIG. 1, adapted from Table 1 of Rabbitts, T., 1994, Nature 372:143-149. The list may be supplemented by additional information found, e.g., in Rowley, J, 1999, Semin. Hematol. 36:59-72 and other publications known in the art, as well as discussion below.

Myeloid cancers in particular are within the scope of the invention and include chromosomal abnormalities that give rise to oncogenic fusion proteins that drive the growth of chronic myeloid leukemia (CML), chronic myelomonocytic leukemia (CMML), acute myeloid leukemia (AML), acute promyelocytic leukemia (APL), and acute lymphoblastic leukemia (ALL). The following chromosomal aberrancies give rise to some illustrative fusions implicated in various forms of ALL:

t(1:19)(q23:p13) Pro-pre-B acute lymphoblastic leukemia

t(12:21)(p13:q32) Pro-pre-B acute lymphoblastic leukemia

t(9:22)(q34:q11) B or B-myeloid acute lymphoblastic leukemia

t(9:12)(q34:p13) Acute B-lymphoblastic leukemia

del(12p) Acute B-lymphoblastic leukemia

Specific genes and proteins thereof implicated in various ALL forms include the MLL gene and the TEL gene, which are commonly rearranged in tumors. Rowley, J, supra. Each has numerous fusion partners. ETV6 denotes the name of the TEL gene product. Fusion of TEL/ETV6 to an acyl CoA synthetase, ACS2, results from a t(5;12)(q31;p13) AML event (Yagasaki, F et al, 1999, Genes Chromosomes Cancer 26:192-202); fusion of TEL/ETV6 to ABL-related gene (ARG) results from a t(1;12)(q25;p13) AML event (Iijima, Y et al, 2000, Blood 95:2126-2131); fusion of TEL/ETV6 to the neurotrophin-3 receptor TRKC results from a t(12;15)(p13;q25) AML event and gives rise to congenital fibrosarcoma (Liu, Q et al, 2000, EMBO J. 19:1827-1838, Eguchi, M et al, 1999, Blood 93:1355-1363); fusion of TEL/ETV6 to the aryl hydrocarbon receptor ARNT results from a t(1;12)(q21;p 13) event and gives rise to acute myeloblastic leukemia (AML-M2) (Salomon-Nguyen, F et al, 2000, Proc. Natl. Acad. Sci. 97:6757-6762); and fusion of TEL/ETV6 to AML-1, the DNA-binding subunit of the AML-1/CBFb transcription factor results from a (12;21)(q13;p32) event that can give rise to acute lymphoblastic leukemia (ALL, Shurtleff, S A et al, 1995, Leukemia 9:1985-1989) and, in some cases, non-Hodgkin's lymphoma (NHL).

Another illustrative fusion within the scope of the invention is the EWS/FLI-1 hybrid protein that is the hallmark of Ewing's sarcoma and the primitive neuroectodermal tumor family (Silvany, et al, 2000, Oncogene 19:4523-4530).

Yet another illustrative family of fusion proteins within the scope of the invention is the group of fusion proteins arising from chromosomal rearrangements involving the RET gene in thyroid cancer (Kolibaba, K, et al, 1997, Biochem. Biophys. Acta 1333:F217-F248). Rearrangements of RET, resulting in juxtaposition of the RET tyrosine kinase domain with one of three 5′ sequences (RET-PTC-1, -2 and -3) generate fusion proteins comprising the kinase domain of RET fused to parts of the genes H4 (RET-PTC-1), R1a of cAMP-dependent protein kinase A (RET-PTC-2) and ELE-1 (RET-PTC-3).

The scope of the present invention also includes cancers and other proliferative diseases, e.g., rheumatoid arthritis, now known or discovered in the future to be characterized by specific chromosomal aberrations giving rise to fusion proteins.

In at least some cases, heterogeneity of breakpoints within the affected chromosomes is possible, thus providing for the possibility of many different DNA fusions and amino acid sequence variations than those specifically listed in the SEQ ID NOs provided, and which can also be formed by the chromosomal rearrangements, e.g., translocations, inversions, deletions, insertion/duplications, etc., so designated. For example, many different abl-bcr gene combinations and corresponding fusion proteins can be designated by the t(9; 22)(q34; q11) translocation event, and all—not just those listed below—are included within the purview of the designation, t(9;22)(q34;q11).

Aberrant proteins of the invention, at least in some instances, feature one or more properties of the individual normal parent genes' gene products (normal polypeptide gene product(s), including e.g., functional and structural domains and subportions thereof resulting from transcription and translation of normal parent genes on normal chromosomes) but otherwise lack exact identity and function with the parent genes' protein products. Chromosomal aberrations may give rise to in-frame fusions or frame-shifts, the latter of which can account for missense or nonsense translation of at least a portion of the mRNA, and thereby result in aberrant polypeptide product(s).

Of the SEQ ID NOs discussed herein, some reflect fusion genes, some reflect fusion gene products, e.g., mRNAs and peptides, and some reflect portions of such entities. Still some others reflect recombination “hot spots” in the normal genes that have a general propensity to form a chromosomal aberration. Each of the above sequences may be useful as diagnostic markers in appropriate embodiments of the invention and/or may be characteristic of a given proliferative disorder (or patient exhibiting such and, accordingly, a candidate for treatment according to some methods of the invention.

While the specific sequences discussed are predominantly human in origin, it is understood that other animal “homologs” of the corresponding human sequences are known in the art and are intended to be within the purview of various aspects of the invention. Because HSP90s are also found in plants, plants and plant cells and tissues exhibiting fusion protein products that give rise to undesirable traits may also be treatable in some aspects and embodiments of the invention. The NCBI nucleotide and protein databases are an example of where such sequences can be found. It is also appreciated that the complete human genome and other genomes have been sequenced, and continue to be sequenced at a high rate, thus facilitating the identity of sequences contiguous with those listed herein and homologs thereto.

Further, some of the sequences listed herein may contain errors associated with the logistical complexities of compiling such extensive data, and the true sequences should be interpreted to be within the scope of the invention, either literally or under the doctrine of equivalents, as they are known in the art.

As those of ordinary skill will appreciate, allelic variations and different isotype proteins are also possible for some genes, e.g., the product of differential splicing events in mRNA, and these are likewise considered within the scope of the invention. Further, some of the NCBI and SEQ ID NOs listed below are for wild-type genes, and are included to give an indication of the different chimeric possibilities for the fused counterpart during a chromosomal aberration according to the invention. Should any of the sequences listed below be in error, such should be construed consistent with what is commonly understood in the art-irrespective of how presented in the application.

c. Further Discussion of Illustrative Chromosomal Aberrancies

Convention: where two or more SEQ ID NOs are provided per NCBI accession #, peptide(s) shall be listed first where applicable, followed by corresponding mRNA/cDNA and/or genomic sequence as the case may be. The terms “nucleotide” and “nucleotides” are interchangeable with, and may be symbolized by, “nt.”

t(9; 22)(q34; q11)

This translocation is generally addressed in FIG. 1. Illustrative embodiments include but are not limited to events comprising the sequences:

NCBI # S72478, corresponding to SEQ ID NOs 1 and 2, illustrates one aberrant polypeptide/mRNA in a patient having CML and another patient having ALL. The junction for the nucleic acid sequence between the BCR and ABL genes is stated to reside between nucleotides 100 and 101., with 1-100 derived from BCR and 101-140 derived from ABL.

NCBI #M19695 (SEQ ID NO 3) illustrates a nucleic acid sequence identified from a human myelocytic chimeric bcr/chromosome 9 fusion (CML K562 cell line).

NCBI #M30829 (SEQ ID NOs 4 and 5) illustrates a partial bcr/abl fusion protein mRNA.

NCBI #M13096 (SEQ ID NO 6) illustrates a human chimeric bcr/c-abl fusion protein gene characteristic of cell line K562.

NCBI #M30832 (SEQ ID NOs 7 and 8) corresponds to a human bcr/abl fusion protein, partial cds, clone E3 from cell line EM2.

NCBI # AJ131466 (SEQ ID NOs 9 and 10) corresponds to a partial human bcr/abl (major breakpoint) fusion peptide and the underlying nucleic acid encoding it. Nucleotides 1-373 are said to derive from exons 11-14 of the bcr gene, and nucleotides 374-997 are said to derive from exons 2-4 of the abl gene.

NCBI # AF192533 (SEQ ID NOs 11 and 12) corresponds to a partial human bcr/abl (major breakpoint) fusion mRNA. Nucleotides 1-289 are said to come from the bcr gene of chromosome 22 and nucleotides 290-305 from the able gene of chromosome 9.

NCBI # AF321981 (SEQ ID NO 13) corresponds to a BCR-ABL fusion transcript e15a2 mRNA sequence. This particular fusion is stated to result from results from a translocation between the 3′ portion of the c-ABL oncogene on chromosome 9 and exon 15 of the BCR gene on chromosom22; t(9;22).

NCBI # M17543 (SEQ ID NO 14) corresponds to at least a portion of a Philadelphia chromosome breakpoint cluster region associated with one embodiment of a bcr abl fusion gene. Nucleotides 1-31 are said to be exon 1 and nucleotides 32-63 are said to be intron A.

NCBI # M17542 (SEQ ID NOs 15 and 16) corresponds to a human bcr/abl fusion protein mRNA (product of translocation t(22q11; 9q34)), exons 1 and 2. Nucleotides 1-31 are stated to denote exon 1 and nucleotides 32-63 are stated to denote exon 2.

NCBI # M17541 (SEQ ID NOs 17 and 18) corresponds to a human bcr/abl fusion protein mRNA (product of translocation t(22q11; 9q34)), exons 1 and 2. Nucleotides 1-31 are stated to denote exon 1 and nucleotides 32-63 are stated to denote exon 2.

NCBI # AB069693 (SEQ ID NOs 19 and 20) denotes a human partial mRNA corresponding to a bcr/abl e8a2 fusion protein. BCR exons 7 (nucleotides 1-53) and 8 (nucleotides 54-194) are joined to ABL intron1b inverted (nucleotides 195-249) and ABL exon a2 (nucleotides 250-423).

NCBI # AJ131467 (SEQ ID NOs 21 and 22) correspond to a human partial BCR/ABL chimeric fusion peptide and corresponding mRNA. Nucleotides 1-117 denote exon 1 of the bcr gene, nucleotides 118-193 and 194-298 denote exons 12 and 13 of the bcr gene, and nucleotides 299-472, 473-768, and 769-922 respectively denote exons 2-4 of the abl gene.

NCBI # AF113911 (SEQ ID NOs 23 and 24) correspond to a partial BCR-ABL minor breakpoint peptide (BCR-ABL fusion) mRNA. Nucleotides 1-455 are stated to be from chromosome 22 and nucleotides 456-1079 from chromosome 9.

NCBI # AF251769 (SEQ ID NOs 25 and 26) correspond to a human partial bcr/abl e1-a3 chimeric fusion protein (13CR/ABLe1-a3) mRNA. Nucleotides 1-455 are stated to be from chromosome 22 and nucleotides 456-1079 from chromosome 9.

inv14 (q11; q32)

This translocation is generally addressed in FIG. 1. Illustrative embodiments include but are not limited to events comprising the sequences:

NCBI # X82240 (SEQ ID NOs 27 and 28) correspond to at least a portion of an mRNA for the gene TCL1, which is disrupted in aberrations of the type noted.

NCBI # NM_{—}021966 (SEQ ID NOs 29 and 30) relate to a human T-cell leukemia/lymphoma 1A (TCL1A), mRNA.

NCBI # X82241 (SEQ ID NO 31) relates to a 5′ portion of a human TCL1 gene. Nucleotides 496-560 are said to correspond to exon 1.

NCBI # M14198 (SEQ ID NOs 32 and 33) relate to a human chromosome 14 paracentric inversion producing an heavy chain/T-cell receptor J-alpha fusion protein.

NCBI # X03752 (SEQ ID NOs 34 and 35) relate to a human gene for rearranged Ig V(H) are said to encode the IgVH region (108 aa) and nucleotides 324 to 377 are said to encode 18 amino acids of the TCR-J-alpha protein.

NCBI # M12071 (SEQ ID NOs 36 and 37) relates to a human Ig heavy-chain V-region gene (VII family) rearranged to T-cell receptor alpha-chain D-J-sp region (IgT) in an inv(14)(q11; q32), SUP-T1 cell line. Nucleotides 121-166 are said to derive from exon 1 of the IgH gene, nucleotides 167-248 from intron 1 of the IgH gene, nucleotides 249-623 from exon 2 of the IgH gene, and nucleotides 624-675 from intron 2 of the IgH gene.

NCBI # S45947 (SEQ ID NOs 38 and 39) relate to an IgT=T cell specific exon ET-immunoglobulin VH-T cell receptor J alpha fusion [human, T cell lymphoma cell line SUP-T1, mRNA Mutant, 508 nt]. Nucleotides 34-507 are stated to be IgT coding sequence.

NCBI # S45207 (SEQ ID NOs 40 and 41) relate to an IgT=T cell specific exon ET-exon EX-immunoglobulin VH-T cell receptor J alpha fusion [human, T cell lymphoma cell line SUP-T1, mRNA Mutant, 616 nt]. Nucleotides 130-616 are stated to be IgT coding sequence.

t(1; 19)(q23; p13.3)

This translocation is generally addressed in FIG. 1. Illustrative embodiments include but are not limited to events comprising the sequences:

NCBI # M31522 (SEQ ID NOs 42 and 43) relate to a human translocation (t1;19) fusion protein (E2A/PRL) mRNA, 3′ end.]. Nucleotides 1-1653 are stated to encode a portion of an E2A/PRL fusion protein.

t(17; 19)(q22; p13)

This translocation is generally addressed in FIG. 1. Illustrative embodiments include but are not limited to events comprising the sequences:

NCBI # M95586 (SEQ ID NOs 44 and 45) relate to a human E2A/HLA fusion protein (E2A/HLF) mRNA, complete cds. Nucleotides 31-1755 are said to be coding sequence.

t(15; 17)(q21-q11-22)

This translocation is generally addressed in FIG. 1. Illustrative embodiments include but are not limited to events comprising the sequences:

NCBI # S50916 (SEQ ID NOs 46 and 47) relate to a PML-RAR fusion gene {fusion transcript} [human, mRNA Partial, 1284 nt]. Nucleotides 1-1251 are said to be coding sequence.

NCBI # M73779 (SEQ ID NOs 48 and 49) relate to a human PML-RAR protein (PML-RAR) mRNA, complete cds; coding sequence: nucleotides 67-2460.

NCBI # AJ417079 (SEQ ID NOs 50 and 51) relate to a human partial mRNA for PML/RARA fusion protein (PML/RARA gene); Nucleotides 1-109 derive from exon 6 of PML, nucleotides 110-172 from intron 2 of RARA, and nucleotides 173-296 from exon 3 of RARA.

t(11; 17)(q23; q21.1)

This translocation is generally addressed in FIG. 1. Illustrative embodiments include but are not limited to events comprising the sequences:

NCBI # AAB29813 (SEQ ID NO 52) relates to a retinoic acid receptor alpha, RAR alpha(PLZF=zinc finger protein, PLZF-RAR alpha isoform A=fusion protein) {translocation} [human, acute promyelocytic leukemia patient, Peptide Mutant, 858 aa].

NCBI # AAB29814 (SEQ ID NO 53) relates to a PLZF=zinc finger protein(retinoic acid receptor alpha, RAR alpha, RAR alpha 1-PLZF isoform B=fusion protein) {translocation} [human, acute promyelocytic leukemia patient, Peptide Mutant, 277 aa].

t(4; 11)(q21; q23)

This translocation is generally addressed in FIG. 1. Illustrative embodiments include but are not limited to events comprising the sequences:

NCBI # L22179 (SEQ ID NOs 54 and 55) relate to a human MLL-AF4 der(11) fusion protein mRNA, complete cds. Nucleotides 5-6940 are said to be coding sequence.

NCBI # S67825 (SEQ ID NOs 56 and 57) relate to a human ALL1-AF4 fusion protein mRNA, partial cds. Nucleotides 1-585 are said to derive from chromosome 11 and nucleotides 586-832 from chromosome 4.

NCBI # AF024541 (SEQ ID NOs 58 and 59) relate to a human MLL-AF4 fusion protein mRNA, partial cds. The codons are said to start with nucleotide 3.

NCBI # AF031404 (SEQ ID NOs 60 and 61) relate to a human MLL-AF4 fusion protein mRNA, partial cds. Nucleotides 1-305 are said to derive from chromosome 11 and nucleotides 306-741 from chromosome 4. Codons begin with nucleotide 3.

NCBI # L04731 (SEQ ID NO 63) relates to a human translocation T(4:11) of the human ALL-1 gene to chromosome 4.

NCBI # AF177237 (SEQ ID NOs 64 and 65) relate to human cell-line MV4-11, MLL/AF4 fusion protein (MLL/AF4) mRNA, partial cds. Nucleotides 1-62 derive from exon 6 of the MLL gene on chromosome 11, and nucleotides 63-450 from exon 5 of the AF4 gene on chromosome 4.

NCBI # AF177236 (SEQ ID NOs 66 and 67) relate to a human A1 MLL/AF4 fusion protein (MLL/AF4) mRNA, partial cds. Nucleotides 1-63 are stated to derive from exon 6 of the MLL gene on chromosome 11, and nucleotides 64-450 from exon 5 of the AF4 gene on chromosome 4.

NCBI # AF031403 (SEQ ID NO 68) relates to a human MLL/AF4 translocation breakpoint t(4;11)(q21;23). Nucleotides 1-105 are said to derive from exon 5 of MLL, nucleotides 435-508 from exon 6 of MLL, nucleotides 2195-2326 from exon 7 of MLL, nucleotides 2874-2987 from exon 8 of MLL, and nucleotides 3645-6983 from AF4.

NCBI # AF177238 (SEQ ID NOs 69 and 70) relate to a human A1 AF4-MLL fusion protein (AF4-MLL) mRNA, partial cds. Nucleotides 1-484 are said to derive from exon 3 of AF4 and nucleotides 485-596 from exon 7 of MLL.

NCBI # AF177239 (SEQ ID NOs 71 and 72) relate to a human cell-line MV4-11 AF4-MLL fusion protein (AF4-MLL) mRNA, partial cds. Nucleotides 1-484 are said to derive from exon 3 of AF4 and nucleotides 485-596 from exon 7 of MLL

NCBI # AF397907 (SEQ ID NO 73) relates to a human AF4/MLL translocation breakpoint region. Nucleotides 1-437 are said to derive from intron 3 of AF6, nucleotides 440-631 from intron 6 of MLL, and nucleotides 632-747 from exon 7 of MLL. The breakpoint is approximately nucleotide 438-439, which was undetermined due to GC compressions.

NCBI # AF024543 (SEQ ID NO 74) relates to a human MLL/AF4 translocation breakpoint t(4;11)(q21;q23).

t(9; 11)(q21; q23)

This translocation is generally addressed in FIG. 1. Illustrative embodiments include but are not limited to events comprising the sequences:

NCBI # S82034 (SEQ ID NO 75) relates to an MLL-AF9=fusion gene {fusion site} [human, peripheral blood, acute myeloid leukemia FAB type M1 patient UPN 427, mRNA Partial, 60 nt].

t(11; 19)(q23; p13)

This translocation is generally addressed in FIG. 1. Illustrative embodiments include but are not limited to events comprising the sequences:

NCBI # S81007 (SEQ ID NO 76) relates to an MLL/ENL=fusion gene {rearranged derivative 11 junction region} [human, leukemic lymphoblasts, T-cell acute lymphoblastic leukemia patient RUPN2, Genomic Mutant, 74 nt]. The authors indicated that the first 34 nt derived from MLL intron 8 on 11q23, and nt 35-74 from the ENL-distal region on 19p13.3

NCBI # S81008 (SEQ ID NO 77) relates to an ENL {rearranged derivative 19 junction region} [human, leukemic lymphoblasts, T-cell acute lymphoblastic leukemia patient RUPN2, Genomic Mutant, 84 nt]. The authors indicated that nt 55-84 derived from MLL gene 3′ region on 11q23.

t(X; 11)(q13; q23)

This translocation is generally addressed in FIG. 1. Illustrative embodiments include but are not limited to events comprising the sequences:

NCBI # NM_{—}005938 (SEQ ID NOs 78 and 79) relate to a human myeloid/lymphoid or mixed-lineage leukemia (trithorax homolog, Drosophila); translocated to, 7 (MLLT7), mRNA. Nucleotides 183-1688 denote an MLLT7 coding region, with nucleotides 465-719 and 480-749 corresponding to a forkhead and forkhead domain, and G and C allelic variations possible at nucleotide 1435.

NCBI # X93996 (SEQ ID NOs 80 and 81) relate to a human mRNA for AFX protein. Nucleotides 183-1688 are said to be AFX coding sequence.

t(1; 11)(p32; q23)

This translocation is generally addressed in FIG. 1. Illustrative embodiments include but are not limited to events comprising the sequences:

NCBI # AF331760 (SEQ ID NO 82) relates to human clone UPN5379L mRNA sequence (bone marrow acute lymphoblastic FAB L2 type).

t(6; 11)(q27; q23)

This translocation is generally addressed in FIG. 1. Illustrative embodiments include but are not limited to events comprising the sequences:

NCBI # S82519 (SEQ ID NOs 83 and 84) relate to a human MLL-AF6 fusion protein mRNA, partial cds, identified in a leukemic patient, and with the breakpoint stated to be approximately between nt 26 and 27.

NCBI # S82521 (SEQ ID NOs 85 and 86) relate to a an MLL-AF6=fusion gene {breakpoint region, clone b} [human, blood, leukemic patient 2, mRNA Partial, 69 nt]. The breakpoint here is said to reside between nt 24 and 25.

NCBI # S82517 (SEQ ID NOs 87 and 88) relate to an MLL-AF6=fusion gene {breakpoint region} [human, blood, leukemia patient 1, mRNA Partial, 69 nt]. The breakpoint here is said to reside between nt 24 and 25.

t(11; 17)(q23; q21)

This translocation is generally addressed in FIG. 1. Illustrative embodiments include but are not limited to events comprising the sequences:

NCBI # S72604 (SEQ ID NOs 89 and 90) relate to an AF17 . . . ALL-1 {reciprocal translocation} [human, acute myeloid leukemia patient, mRNA Partial Mutant, 3 genes, 228 nt]. Nucleotides 1-88 are said to derive from AF17 and nucleotides 89-228 from ALL-1.

NCBI # (SEQ ID NOs 91 and 92) relate to a human myeloid/lymphoid or mixed-lineage leukemia (trithorax homolog, Drosophila); translocated to, 6 (MLLT6), mRNA. Nucleotides approximating 22-168 are said to encode a PHD zinc finger motif and nucleotides 2185-2292 (amino acids 729-764) are said to encode a leucine zipper motif, with A and G allelic variations at nt 592 possible.

t(8; 21)(q22; q22)

This translocation is generally addressed in FIG. 1. Illustrative embodiments include but are not limited to events comprising the sequences:

NCBI # (SEQ ID NOs 93 and 94) relate to a human mRNA for AML1-MTG8 fusion protein, complete cds. The coding sequence is said to be nucleotides 1579-3837 and the breakpoint is said to be between nt 2110 and 2111.

NCBI # S78158 (SEQ ID NOs 95 and 96) relate to a human AMLI-ETO fusion protein (AML1-ETO) mRNA, partial cds. Nucleotides 1-1767 are said to denote the coding sequence.

NCBI # S78159 (SEQ ID NOs 97 and 98) relate to a human AML1-ETO fusion protein (AML1-ETO) mRNA, partial cds. Nucleotides 1-696 are said to denote the coding sequence and nucleotides 40 and 41 are said to represent the junction point.

NCBI # D14822 (SEQ ID NOs 99 and 100) relate to a human chimeric partial mRNA derived from AML1 and MTG8(ETO) gene sequences. Nucleotides 1-101 are said to derive from the AML1 gene on chromosome 21 and nucleotides 102-799 from the MTG8 (ETO) gene on chromosome 8.

NCBI # S45790 (SEQ ID NO 101) relates to a AML1/ETO=acute myelogenous leukemia {translocation breakpoint} [human, Genomic Mutant, 237 nt].

NCBI # Z35296 (SEQ ID NO 102) relates to a human AML1/ETO alternative fusion transcript mRNA, 276 bp. Nucleotides 1-117 are said to derive from AML1 and 186-276 are said to derive from ETO.

NCBI # D14823 (SEQ ID NOs 103 and 104) relate to a human chimeric mRNA derived from AML1 gene and MTG8(ETO) gene, partial sequence. Nucleotides 1-101 are said to be derived from the AML1 gene on chromosome 21 and nucleotides 102-1446 are said to be derived from the MTG8(ETO) gene on chromosome 8, with the coding sequence denoted nt 1-757.

t(3; 21)(q26; q22)

This translocation is generally addressed in FIG. 1. Illustrative embodiments include but are not limited to events comprising the sequences:

NCBI # S69002 (SEQ ID NOs 105 and 106) relate to a AML1-EVI-1=AML1-EVI-1 fusion protein {rearranged translocation} [human, leukemic cell line SKH1, mRNA Mutant, 5938 nt]. The author indicated the boundary between AML1 and EVI-1 to be between nt 2138 and 2139, with the coding sequence being 1603-5790.

NCBI # L21756 (SEQ ID NOs 107 and 108) relate to a human acute myeloid leukemia associated protein (AML1/EAP) mRNA, complete cds. Nucleotides 1-786 are said to denote the coding sequence.

NCBI # S76343 (SEQ ID NO 109) relates to AML1 . . . EAP {translocation breakpoint} [human, chronic myelogenous leukemia in blast crisis patient, Genomic Mutant, 3 genes, 470 nt]. Nucleotides 1-125 are said to derive from AML1 and nucleotides 126-470 are said to derive from EAP.

t(16; 21)(p11; q22)

This translocation is generally addressed in FIG. 1. Illustrative embodiments include but are not limited to events comprising the sequences:

NCBI # S71718 (SEQ ID NOs 110 and 111) relate to a TLS/FUS . . . ERG {translocation} [human, myeloid leukemia patient, peripheral blood, bone marrow cells, mRNA Partial Mutant, 3 genes, 55 nt]. Nucleotides 46-55 are said to derive from ERG, with the codon start beginning with nt 3.

NCBI # S71805 SEQ ID NOs 112 and 113) relate to a TLS/FUS . . . ERG {translocation} [human, myeloid leukemia patient, peripheral blood, bone marrow cells, mRNA Partial Mutant, 3 genes, 99 nt]. Nucleotides 1-89 are said to derive from TLS/FUS and nucleotides 90-99 from ERG, with the codon start beginning with nt 3.

NCBI # Y10001 (SEQ ID NO 114) relates to a DNA fragment containing fusion point of FUS gene and ERG gene, translocation t(16;21)(p11;q22).

t(6; 9)(p23; q34)

NCBI # X64229 (SEQ ID NOs 115 and 116) relate to a human dek mRNA. The coding sequence is said to be nt 34-1161.

inv(9;9)

This translocation is generally addressed in FIG. 1. Illustrative embodiments include but are not limited to events comprising the sequences:

NCBI # X63689 (SEQ ID NO 117) relates to a human translocation breakpoint in the “can” gene sequence. The translocation breakpoint is said to be 174.175.

NCBI # M93651 (SEQ ID NOs 118 and 119) relate to a human set gene, complete cds. The coding sequence is said to be 4-837.

t(4; 16)(q26; p13)

This translocation is generally addressed in FIG. 1. Illustrative embodiments include but are not limited to events comprising the sequences:

NCBI # Z14955 (SEQ ID NOs 120 and 121) relate to a human mRNA encoding the interleukin 2/BCM fusion protein. Nucleotides 1-321 derive from exons 1-3 of IL-2 and nucleotides 322-864 from the BCM gene.

inv(16)(p13q22)

This inversion is generally addressed in FIG. 1. Illustrative embodiments include but are not limited to events comprising the sequences:

NCBI # AF251768 (SEQ ID NOs 122 and 123) relate to a human PCBFB/MYH11E chimeric fusion protein (CBFB/MYH11) mRNA, partial cds. Nucleotides 1-41 correspond to exon 5 of CBFB and nucleotides 42-78 to exon 7 of MYH11.

NCBI # AF249898 (SEQ ID NOs 124 and 125) relate to a human PCBFbeta/MYH11A chimeric fusion protein (CBFbeta/MYH11A) mRNA, partial cds. Nucleotides 1-41 correspond to exon 5 of CBFB and nucleotides 42-102 to exon 12 of MYH11.

NCBI # AF249897 (SE ID NOs 126 and 127) relate a human PCBFb-MYH11d chimeric fusion protein (CBFB/MYH11D) mRNA, partial cds. Nucleotides 1-41 correspond to exon 5 of CBFB and nucleotides 42-109 to exon 8 of MYH11.

NCBI # AF390860 (SEQ ID NO 128) relates to a human isolate UPN2 CBFB/MYH11 translocation breakpoint region sequence.

NCBI # AF390859 (SEQ ID NO 129) relates to a human isolate UPN1 CBFB/MYH11 translocation breakpoint region sequence.

NCBI # AF202996 (SEQ ID NOs 130 and 131) relate to human core binding factor beta-smooth muscle myosin heavy chain fusion protein (CBFB-MYH11) mRNA, partial cds. Nucleotides 1-46 are said to correspond to 16q22 and nucleotides 47-89 to 16p 13. Nucleotide 50 is said to be a “t” in some cases.

t(5; 12)(q33; p13)

This translocation is generally addressed in FIG. 1. Illustrative embodiments include but are not limited to events comprising the sequences:

NCBI # NM_{—}001987 (SEQ ID NOs 132 and 133) relate to a human ets variant gene 6 (TEL oncogene) (ETV6), mRNA. Nucleotides 25-1383 are said to correspond to coding sequence, of which nt 136-393 are said to correspond to a sterile alpha motif (SAM) pointed domain, nt 1036-1290 to an erythroblast transformation-specific (Ets)-domain, and wherein allelic variations including “c”s and “t”s at each of nt 798, nt 1541, and nt 1598, and an “a”s and “c”s at each of nt 1822 and 1881.

NCBI # U11732 (SEQ ID NOs 134 and 135) relate to a human ets-like gene (tel) mRNA, complete cds. The coding sequence is said to be from nt 25-1383, and the translocation breakpoint said to occur after nt 487.

t(2; 5)(2p23; q35)

This translocation is generally addressed in FIG. 1. Illustrative embodiments include but are not limited to events comprising the sequences:

NCBI #14: AF032882 (SEQ ID NO 136) relates to a human anaplastic lymphoma kinase receptor (ALK) and nucleophosmin (NPM) truncated genes at a t(2;5) translocation breakpoint. Nucleotides 1-46 are said to be ALK sequence that is truncated at 3′ due to translocation, and nucleotides 1370-1451 are said to be NPM sequence that is truncated at 5′ due to translocation.

NCBI # S82740 (SEQ ID NO 137) relates to a NPM/ALK=fusion gene {translocation breakpoint} [human, lymphoma cells SUP-M2, Genomic, 1565 nt].

NCBI # S82725 (SEQ ID NO 138) relates to a NPM/ALK=fusion gene {translocation breakpoint} [human, lymphoma cells SU-DHL-1, Genomic, 1679 nt].

NCBI # U04946 SEQ ID NOs 139 and 140) relate to a human nucleophosmin-anaplastic lymphoma kinase fusion protein (NPM/ALK) mRNA, complete cds. The recombination junction is said to occur at nt 353.

t(11; 22) (q24; q12)

This translocation is generally addressed in FIG. 1. Illustrative embodiments include but are not limited to events comprising the sequences:

NCBI # AJ229320 (SEQ ID NO 141) relates to a human translocation t(11;22) DNA in ewings's tumor derivative 22 (isolate: EWTUM64/MIC). Nucleotides 1-88 are said to denote EWS sequence and nucleotides 89-180 FLI-1 sequence.

NCBI # AJ229311 SEQ ID NO 142) relates to a human translocation t(11;22) DNA in ewings's tumor derivative 22 (isolate: EWTUM56/EW20). Nucleotides 1-114 are said to denote EWS sequence and nucleotides 115-180 FLI-1 sequence.

NCBI # AF177752 (SEQ NO 143) relates to a human clone Jugo Ewing's sarcoma-specific EWS-FLI1 chimera target sequence.

NCBI # AF177751 (SEQ ID NO 144) relates to a human Juyon Ewing's sarcoma-specific EWS-FLI1 chimera target sequence.

NCBI # AF177750 (SEQ ID NO 145) relates to a human clone Iti Ewing's sarcoma-specific EWS-FLI1 chimera target sequence.

NCBI # AF327066 SEQ ID NOs 146 and 147) relate to a human Ewings sarcoma EWS-Fli1 (type 1) oncogene mRNA, complete cds.

NCBI # XM_{—}060745 (SEQ ID NOs 148 and 149) relate to a human similar to EWS/FLI1 activated transcript 2 (H. sapiens) (LOC127935), mRNA. Nucleotides 10-225 and 13-195 are said to denote src homology 2 (SH2) domains.

NCBI # AF403-479 SEQ ID NOs 150 and 151) relate to a human EWS/FLI1 activated transcript 2 protein mRNA, complete cds.

NCBI # AF020264 (SEQ ID NOs 152 and 153) relate to a human EWS/FLI1 activated transcript 2 homolog (EAT-2) gene, partial cds.

NCBI # AF020263 (SEQ ID NOs 154 and 155) relate to a Mus musculus EWS/FLI1 activated transcript 2 (EAT-2) mRNA, complete cds.

NCBI # S72620 SEQ ID NOs 156 and 157) relate to a EWS . . . Fli1 [human, T93-113 tumor, mRNA Partial Mutant, 3 genes, 229 nt]. Nucleotides 1-85 are said to denote partial EWS gene sequence and nt 86-229 are said to denote partial FLI-1 sequence.

NCBI # S64709 (SEQ ID NO 158) relates to EWS . . . Fli-1 {translocation} [human, IARC-EW11 Ewing's tumor-derived cells, mRNA Mutant, 3 genes, 100 nt]. Nucleotides 1-18 are said to denote partial EWS gene sequence and nt 19-100 are said to denote partial FLI-1 sequence.

NCBI # S62665 (SEQ ID NOs 159 and 160) relate to a type 4 EWS-FLI1 fusion {translocation} [human, primitive neuroectodermal tumor cell line TC-32, mRNA Partial Mutant, 60 nt]. Positions 1-31 are said to be from the 5′ portion of EWS on chromosome 22 and positions 32-60 are said to be from the 3′ (DNA-binding) region of FLI1 on chromosome 11.

inv(10)(q11.2; q21)

This aberration is generally addressed in FIG. 1. Illustrative embodiments include but are not limited to events comprising the sequences:

NCBI # AF395885 (SEQ ID NO 161) relates to a human H4/RET fusion mRNA, partial sequence. tyrosine kinase domain of the ret. Nt 1-83 are said to derive from H4, nt 84-142 from an unidentified insertion sequence, and nt 143-447 from ret. The tyrosine kinase domain in the ret portion is said be constitutively active in the fusion product.

NCBI # NM_{—}005436 (SEQ ID NOs 162 and 163) relate to a human DNA segment, single copy, probe pH4 (transforming sequence, thyroid-1, (D10S170), mRNA. Nt 37-1794 are said to represent coding sequence, nt 202-996 said to encode a mysosin tail, nt 610-999 an Ezrin/radixin/moesin family (ERM) region, with “a” and “c” allelic variation possible at nts 979, 1080, and 1445, and “a” and “g” possible at nt 1362, and “t” and “c” possible at nts 1996 and 2642.

NCBI # S77910 (SEQ ID NO 164) relates to H4=gene frequently rearranged with the ret proto-oncogene {promoter} [human, Genomic, 447 nt]. Nt 442-447 are said to correspond to the coding sequence, “MA”.

NCBI # S72869 (SEQ ID NOs 165 and 166) relate to H4(D10S170)=putative cytoskeletal protein [human, thyroid, mRNA, 3011 nt]. Nt 37-1794 are said to correspond to coding sequence.

NCBI # X65617 (SEQ ID NO 167) relates to a human ret proto-oncogene DNA. Nt 1-54 are said to replace sequences from the H4 gene, nt 55-787 are said to correspond to an intron between the transmembrane and tyrosine kinase domain, and nt 788-808 said to correspond to an exon coding for a tyrosine kinase domain.

t(12;22)(q13;q12)

This translocation is generally addressed in FIG. 1. Illustrative embodiments include but are not limited to events comprising the sequences:

NCBI # NM_{—}005171 (SEQ ID NOs 168 and 169) relate to a human activating transcription factor 1 (ATF1), mRNA. Nt 157-252 are said to correspond to a pKID domain and nt 631-795 are said to correspond to a bZIP transcription factor region.

NCBI # AF047022 (SEQ ID NOs 170 and 171) relate to a human RNA binding protein-activating transcription factor-1 fusion protein (EWS-ATF1) mRNA, partial cds. Nt 1-65 are said to correspond to chromosome 22 and nt 66-353 to chromosome 12, with nt 66ˆ67 said to represent the fusion junction between the EWS and ATF1 genes.

t(12; 16(q13; p11)

This translocation is generally addressed in FIG. 1. Illustrative embodiments include but are not limited to events comprising the sequences:

NCBI # AJ301614 (SEQ ID NO 172) relates to a human t(12;16)(q13;p11) translocation breakpoint (CHOP/FUS chimaeric genomic DNA). Nt 1-225 are said to correspond to the CHOP gene (chromosome 12) and nt 226-500 to the FUS gene (chromosome 16).

NCBI # AJ301613 (SEQ ID NO 173) relates to a human t(12;16)(q13;p11) translocation breakpoint (FUS/CHOP chimaeric genomic DNA). Nt 1-317 are said to correspond to the FUS gene (chromosome 16) and nt 318-521 to the CHOP gene (chromosome 12).

NCBI # AJ301612 (SEQ ID NOs 174 and 175) relate human partial mRNA for FUS/CHOP chimaeric fusion protein (type 9 transcript variant). Nt 1-118 are said to originate from chromosome 16 and nt 119-225 are said to originate from chromosome 12.

NCBI # AJ301611 (SEQ ID NOs 176 and 177) relate to a human partial mRNA for FUS/CHOP chimaeric fusion protein (type 8 transcript variant). Nt 1-128 are said to originate from chromosome 16 and nt 129-235 are said to originate from chromosome 12.

NCBI # NM_{—}004960 (SEQ ID NOs 178 and 179) relate to a human fusion protein derived from t(12;16) malignant liposarcoma (FUS), mRNA. Nt 79-1659 are said to denote the coding sequence. Allelic variation is stated to be possible at nts 225 (a/c), 369 (c/t), and 1586 (a/g). Nt 937-1173 are said to denote an RNA recognition motif (RRM), and nt 1354-1425 are said to denote a zinc finger domain in a Ran binding proteins (zf-Ranbp).

NCBI # S75762 (SEQ ID NOs 180 and 181) relate to a FUS . . . CHOP [human, myxoid liposarcoma specimens, mRNA Partial Mutant, 3 genes, 652 nt]. Nucleotides 1-272 are said to derive from FUS.

NCBI #X71427 (SEQ ID NOs 182 and 183) relate to a human mRNA for FUS-CHOP protein fusion. Nucleotides 70-1458 are said to denote the fusion coding sequence.

NCBI # X71428 (SEQ ID NOs 184 and 185) relate to a human mRNA for FUS glycine rich protein. Nucleotides 73-1650 are said to denote the coding sequence.

NCBI # Y10004 (SEQ ID NO 186) relates to a human DNA fragment containing fusion point of FUS gene and CHOP gene, translocation t(12;16)(q13;p11. The sequence is said to contain 5′-FUS intron 7 sequence and intron 13′ sequence from CHOP.

NCBI # Y10003 (SEQ ID NO 187) relates to a human DNA fragment containing fusion point of FUS gene and CHOP gene, translocation t(12;16)(q13;p11. The sequence is said to contain 5′-FUS intron 7 sequence and intron 13′ sequence from CHOP.

NCBI # Y10002 (SEQ ID NO 188) relates to a human DNA fragment containing fusion point of FUS gene and CHOP gene, translocation t(12;16)(q13;p11). The sequence is said to contain 5′-FUS intron 7 sequence and intron 13′ sequence from CHOP.

NCBI # S75763 (SEQ ID NOs 189 and 190) relate to a FUS . . . CHOP [human, myxoid liposarcoma specimens, mRNA Partial Mutant, 3 genes, 377 nt]. Nt 1-272 are said to derive from FUS and nt 273-377 from CHOP.

t(2; 13)(q35;q14)

This translocation is generally addressed in FIG. 1. Illustrative embodiments include but are not limited to events comprising the sequences:

NCBI # U02308 (SEQ ID NOs 191 and 192) relate a human PAX-3-FKHR gene fusion mRNA, partial cds. Nt 1-2070 are said to be coding sequence.

t(x; 18)(p11.2; q11.2)

This translocation is generally addressed in FIG. 1. Illustrative embodiments include but are not limited to events comprising the sequences:

NCBI # S79894 (SEQ ID NOs 193 and 194) relate to a SYT . . . SSX {translocation breakpoint} [human, synovial sarcoma patient, tumor, mRNA Mutant, 3 genes, 165 nt]. Nt 1-18 are said to derive from SYT and nt 22-165 from SSX.

NCBI # X86175 (SEQ ID NOs 195 and 196) relate to a human mRNA for SSX2 protein. Nt 92-658 are said to be coding sequence.

The following chromosomal aberrations are not discussed in FIG. 1 and will now be discussed in more detail:

t(12:21)(p13:q32)

The TEL (ETV6)-AML1 (CBFA2) gene fusion is the most common reciprocal chromosomal rearrangement in childhood cancer, occurring in approximately 25% of the most predominant subtype of leukemia-common acute lymphoblastic leukemia. Ford et al., Proc. Natl. Acad. Sci. U.S.A. 95 (8), 4584-4588 (1998), reported characterization of the translocation event responsible for one TEL-AML1 genomic sequence in a pair of monozygotic twins diagnosed at ages 3 years, 6 months and 4 years, 10 months with common acute lymphoblastic leukemia. The twins shared an identical rearranged IgH allele. These data have implications for the etiology and natural history of childhood leukemia.

Other articles of interest on this subject include: Wiemels et al., Protracted and variable latency of acute lymphoblastic leukemia after TEL-AML1 gene fusion in utero, Blood. 1999 Aug. 1; 94(3):1057-62; Rubnitz et al., The role of TEL fusion genes in pediatric leukemias, Leukemia, 1999 January; 13(1):6-13. Review; Romana et al., The t(12;21) of acute lymphoblastic leukemia results in a tel-AML1 gene fusion, Blood. 1995 Jun. 15; 85(12):3662-70; Seeger et al., TEL-AML1 fusion in relapsed childhood acute lymphoblastic leukemia, Blood. 1999 94(1):374-6; Bayar et al., Monozygotic twins with congenital acute lymphoblastic leukemia (ALL) and t(4;11)(q21;q23), Cancer Genet Cytogenet. 1996 Jul. 15; 89(2):177-80; Kobayashi et al., Detection of the Der (21)t(12;21) chromosome forming the TEL-AML1 fusion gene in childhood acute lymphoblastic leukemia, Leuk Lymphoma. 1997 December; 28(1-2):43-50; and Shurtleff et al., TEL/AML1 fusion resulting from a cryptic t(12;21) is the most common genetic lesion in pediatric ALL and defines a subgroup of patients with an excellent prognosis, Leukemia, 1995 (12):1985-9.

NCBI# AF044317 (SEQ ID NO 197) relates to a human TEL/AML1 fusion gene, partial sequence. This was derived from an ALL infant. Nts 1-407 are said to derive from TEL and nts 408-548 from AML-1.

NCBI # AF231770 (SEQ ID NO 198) relates to a human ETV6/AML1 translocation breakpoint region.

t(9:12)(q34; p13)

In human leukemia, activation of the ABL proto-oncogene locus on chromosome 9 most commonly occurs as a result of its fusion to the BCR locus on chromosome. Papadopoulos et al., Cancer Res. 55 (1), 34-38 (1995), reported a t(9;12) event—a chimeric ABL protein displaying an elevated tyrosine kinase activity fused to a TEL protein from chromosome 12. Like BCR, TEL is fused in-frame with ABL and produces a fusion protein with an elevated tyrosine kinase activity when assayed in an immune complex. The amino-terminal sequences of TEL encodes a helix-loop-helix motif which may mediate dimerization. 43: See also Okuda et al., Oncogene. 1996 Sep. 19; 13(6):1147-52.

NCBI # Z36279 (SEQ ID NO 199) relates to a human (9TX) breakpoint position DNA for the tel-abl fusion identified by Papadopoulos et al. The translocation breakpoint is said to reside between nt 567 and 568.

del(12p)

Revy et al., Cell 102:565-575 (2000), reported hyper IgM immunodeficiencies associated with deletions of 19 and 9 bases at cDNA positions 21 and 175 respectively of the activation-induced cytidine deaminase (AID) gene. The former results in a 6 amino acid deletions and a phe15 to ter premature nonsense codon. The latter results in a 3-amino acid deletion and leu59-to-phe substitution.

NCBI # AB040430 (SEQ ID NOs 200 and 201) relate to a human AID gene for activation-induced cytidine deaminase, complete cds.

NCBI # AB040431 (SEQ ID NO 202 and 203) relate to a human AID mRNA for activation-induced cytidine deaminase, complete cds. Nt 77-673 is said to be coding sequence.

NCBI # NM_{—}020661 (SEQ ID NOs 204 and 205) relate to a human activation-induced cytidine deaminase (AICDA), in RNA. Nt 77-673 is said to be coding sequence. Allelic variation (a/g) is said to occur at nt 541.

t(15;17)(q22;q12)

de The et al., Cell 1991 Aug. 23; 66(4):675-84, reported a PML-RAR alpha fusion mRNA generated by a t(15; 17) translocation associated with acute promyelocytic leukemia (APL). The gene product contained a novel zinc finger motif common to several DNA-binding proteins and the mRNA encoded a predicted 106 kd chimeric protein containing most of the PML sequences fused to a large part of RAR alpha, including its DNA- and hormone-binding domains. In transient expression assays, the hybrid protein exhibited altered transactivating properties if compared with the wild-type RAR alpha progenitor. Identical PML-RAR alpha fusion points were found in several patients, suggesting that in APL the t(15;17) translocation generates an RAR mutant that could contribute to leukemogenesis through interference with promyelocytic differentiation.

NCBI # S50916 (SEQ ID NOs 206 and 207) relate to a PML-RAR fusion gene {fusion transcript} [human, mRNA Partial, 1284 nt]. Nt 1-1251 is said to be coding sequence.

NCBI # M73779 (SEQ ID NOs 208 and 209) relate to a human PML-RAR protein (PML-RAR) mRNA, complete cds. Nt 67-2460 is said to be coding sequence.

NCBI # AJ417079 (SEQ ID NOs 210 and 211) relate to a human partial mRNA for PML/RARA fusion protein (PML/RARA gene). Nt 1-109 are said to derive from exon 6 of PML and nts 110-172 and 173-296 are said to derive from intron 2 and exon 3 of RARA.

t(11;17)(q23;q12)

Chen et al., EMBO J., 12 (3), 1161-1167 (1993), reported a fusion between a novel Kruppel-like zinc finger gene and the retinoic acid receptor-alpha locus due to a variant t(11;17) translocation associated with acute promyelocytic leukaemia (APL). Chen et al identified mRNAs containing the coding sequences of the new gene, fused in-frame either upstream of the RAR alpha B region or downstream from the unique A1 and A2 regions of the two major RAR alpha isoforms. The new gene, which Chen et al. termed PLZF (for promyelocytic leukaemia zinc finger), encodes a potential transcription factor containing nine zinc finger motifs related to the Drosophila gap gene Kruppel and is expressed as at least two isoforms which differ in the sequences encoding the N-terminal region of the protein. Within the haematopoietic system the PLZF mRNAs are detected in the bone marrow, early myeloid cell lines and peripheral blood mononuclear cells, but not in lymphoid cell lines or tissues. In addition, the PLZF mRNA levels were down-regulated in NB-4 and HL-60 promyelocytic cell lines in response to retinoic acid-induced granulocytic differentiation and were very low in mature granulocytes, suggesting an important role for PLZF as well as retinoic acid and its receptors in myeloid maturation.

NCBI # NM-006006 (SEQ ID NOs 212 and 213) relate to a human zinc finger protein 145 (Kruppel-like, expressed in promyelocytic leukemia) (ZNF145), mRNA. Nt 76-2097 are said to be coding sequence.

NCBI # Z19002 (SEQ ID NOs 214 and 215) relate to a human PLZF gene encoding kruppel-like zinc finger protein. Nt 76-2097 are said to be coding sequence.

t(16:16)(p13;q22) and inv(16)

Springall et al., Leukemia 12 (12), 2034-2035 (1998), identified a novel CBFB-MYH11 fusion transcript in a patient with AML and attributed it to an inversion/translocation of chromosome 16. See also, Kauter et al., Genes Chromosomes Cancer. 2001 April; 30(4):342-8, Detection and quantification of CBFB/MYH11 fusion transcripts in patients with inv(16)-positive acute myeloblastic leukemia by real-time RT-PCR; Martinelli et al., Haematologica. 2000 May; 85(5):552-5, Long-term disease-free acute myeloblastic leukemia with inv(16) is associated with PCR undetectable CBFbeta/MYH11 transcript; and Dierlamm et al., Genes Chromosomes Cancer. 1998 June; 22(2):87-94. Review, FISH identifies inv(16)(p13q22) masked by translocations in three cases of acute myeloid leukemia.

NCBI # AF202996 (SEQ ID NOs 216 and 217) relate to a human core binding factor beta-smooth muscle myosin heavy chain fusion protein (CBFB-MYH11) mRNA, partial cds. Nt 1-46 are said to originate from 16q22 and nt 47-89 are said to originate from 16p13. Nt 50 is said to be a “t” in some reports.

NCBI # AF251768 (SEQ ID NOs 218 and 219) relate to human PCBFB/MYH11E chimeric fusion protein (CBFB/MYH11) mRNA, partial cds. Nt 1-42 are said to derive from exon 5 of CBFB and nts 42-78 from exon 7 of MYHL1.

NCBI # AF249898 (SEQ ID NOs 220 and 221) relate to a human PCBFbeta/MYH11A chimeric fusion protein (CBFbeta/MYH11A) mRNA, partial cds. Nt 1-42 are said to derive from exon 5 of CBFB and nts 42-78 from exon 12 of MYH11.

NCBI # AF249897 (SEQ ID NOs 222 and 223) relate to a human s PCBFb-MYH11d chimeric fusion protein (CBFB/MYH11D) mRNA, partial cds.

NCBI # AF390860 (SEQ ID NO 224) relates to a human UPN2 CBFB/MYH11 translocation breakpoint region sequence.

NCBI # AF390859 (SEQ ID NO 225) relates to a human isolate UPN1 CBFB/MYH11 translocation breakpoint region sequence.

t(9;11)(p22;q23)

Tkachuk et al., Cell 71: 691-700, (1992), showed that the gene involved in recurring 11q23 leukemogenic translocations codes for an unusually large protein that is a homolog of Drosophila ‘trithorax’ and is involved in homeotic gene regulation (MLL; aka ALL1). In studies of a t(11;19) translocation, they identified a chimeric protein containing the amino-terminal ‘AT-hook’ motifs of the MLL gene on chromosome 11 fused to a previously undescribed protein from chromosome 19. The nucleotide sequence determinations demonstrated an open reading frame that coded for a predicted 62-kD protein, which Tkachuk et al. named ENL.

Nakamura et al., Proc. Nat. Acad. Sci. 90: 4631-4635, (1993), showed that the gene on chromosome 19 that is fused to the MLL gene in patients with leukemia and translocation t(11; 19)(q23;p 13) shows high sequence homology to the genes on chromosome 4 and chromosome 9 that are fused with the ALL1 gene in patients with translocation t(4;11)(q21;q23) and t(9;11)(p22;q23), respectively. The 3 protein gene products contained nuclear targeting sequences as well as serine-rich and proline-rich regions. The results suggested that the different proteins fused to ALL1 polypeptides. These leukemias provide similar functional domains.

Negrini et al., Cancer Res 1993 Oct. 1; 53(19):4489-92, reported potential topoisomerase II DNA-binding sites at the breakpoints of a t(9; 11) chromosome translocation in acute myeloid leukemia. The event examined was a t(9;11)(p22;q23) chromosome translocation and the breakpoints on the two chromosomes occurred within introns of the involved genes: AF-9 on chromosome 9, and ALL-1 on chromosome 11. Sequence analysis identified heptamers flanking the breakpoints on both chromosomes 9 and 11, suggesting that the V-D-J recombinase was involved in the translocation. See also Cimino et al., Cancer Res. 1991 Dec. 15; 51(24):6712-4, Cloning of ALL-1, the locus involved in leukemias with the t(4;11)(q21;q23), t(9;11)(p22;q23), and t(11;19)(q23,p13) chromosome translocations.

Poirel et al., Blood 87 (6), 2496-2505 (1996), reported an MLL-AF9=fusion gene {fusion site} [human, peripheral blood, acute myeloid leukemia FAB type M1 patient UPN 427, mRNA Partial, 60 nt]; NCBI # S82034 (SEQ ID NO 226), and indicated the breakpoint to be at nucleotide 29.

t(1;22)(p13;q13)

Nakamura et al., Proc Natl Acad Sci USA 1993 May 15; 90(10):4631-5, correlated aberrations on chromosomes 4, 9, and 19 involved in 11q23 abnormalities in acute leukemia with shared sequence homology and/or common motifs, including fusions of the ENL gene with ALL-1 in (11:19) translocations. ENL proteins contain nuclear targeting sequences as well as serine-rich and proline-rich regions. Stretches abundant in basic amino acids are also present.

NCBI # AF364037 (SEQ ID NOs 227 and 228) relate to a human megakaryoblastic leukemia-1 protein/RNA-binding motif protein 15s+ae fusion protein (MKL1/RBM15 fusion) mRNA, complete cds. Ma et al., Nat. Genet. 28 (3), 220-221 (2001) identified this with an acute megakaryoblastic leukemia patient. Nt 144-221 are said to be coding sequence, with nts 1-150 deriving from chromosome 22 and nts 151-300 deriving from chromosome 1.

t(3;3)(q21;q26) or inv(3)(q21q26)

Ogawa et al., Oncogene 1996 Jul. 4; 13(1):183-91 showed that overexpression of the Evi-1 gene appears to be a consistent feature of the 3q21q26 syndrome, an association of myeloid leukemias/myelodysplastic syndrome with a specific chromosomal aberration involving both 3q21 and 3q26, such as t(3;3)(q21;q26) or inv(3)(q21q26). The rearrangement in 3q26 has been reported to occur near the Evi-1 locus, implicating that it is the critical gene deregulated in the 3q21 q26 syndrome. Ogawa identified a structural abnormality of Evi-1 protein in a case with the 3q21q26 syndrome. That case carried the typical inv(3)(q21q26), in which the 3q26 breakpoint is located within an intron of the Evi-1 gene, and resulted in overexpression of a normally unexpressed, aberrant form of Evi-1 protein, in which the C-terminal 44 amino acids of wild-type Evi-1 protein were truncated and replaced by five amino acids. The truncated Evi-1 protein was shown to increase AP1 activity when expressed in NIH3T3 cells as its wild-type counterpart. The origin of this peculiar type of rearrangement of the Evi-1 gene was shown not to be an artifact during establishment of the cell line, but rather an event that occurred in the primary leukemic cells, and consistent with 3q21q26 syndrome.

NCBI # S82592 (SEQ ID NOs 229 and 230) relate to an Evi-1=Evi-1 protein {3′ region, deletion region} [human, megakaryoblastoid cell line MOLM-1, chronic myelocytic leukemia patient, mRNA Partial Mutant, 916 nt]. Nt 1-132 are said to represent a partial coding sequence.

t(3;5)(q25;q34)

Yoneda-Kato et al., Oncogene 12: 265-275 (1996), showed that t(3;5)(q25.1;q34) of myelodysplastic syndrome and acute myeloid leukemia produces a novel fusion gene, NPM-MLF1, which results from an in-frame fusion between the 5-prime coding region of the nucleophosmin gene on chromosome 5 and a gene on chromosome 3, designated MLF1 (myeloid leukemia factor-1). The translocation was identified in 3 t(3;5)-positive cases of AML. Expression of the mRNA was widespread but highest in testis, ovary, skeletal muscle, heart, kidney and colon. Antibodies to MLF1 detected a 31-kD protein in K562 and HEL erythroleukemia cell lines

NCBI # L49054 (SEQ ID NOs 231 and 232) relate to a t(3;5)(q25.1;p34) fusion gene NPM-MLF1 mRNA, complete cds. Nt 109-915 are said to be coding sequence.

NCBI # BC007045 (SEQ ID NOs 233 and 234) relate to a human myeloid leukemia factor 1, clone MGC:12449, mRNA, complete cds. Nt 107-913 are said to be coding sequence.

NCBI # L49054 (SEQ ID NOs 235 and 236) relate to a human t(3;5)(q25.1;p34) fusion gene NPM-MLF1 mRNA, complete cds. Nt 109-915 are said to be coding sequence.

t(7;11)(p15;p15)

Borrow et al., Nat. Genet. 1996 February; 12(2):159-67, reported a t(7;11)(p15;p15) translocation in acute myeloid leukaemia that fused the genes for nucleoporin NUP98 and class I homeoprotein HOXA9.

NCBI # U41814 (SEQ ID NOs 237 and 238) relate to human NUP98-HOXA9 fusion protein mRNA, partial cds. Nt 46ˆ47 are said to represent a NUP98-HOXA9 in-frame junction and nt 138ˆ139 are said to be an alternative splice site within HOXA9

NCBI # NM_{—}002142 (SEQ ID NOs 239 and 240) relate to a human homeo box A9 (HOXA9), mRNA. Nts 67 and 213 are said to have allelic variation possible (c/g), and nt 397-567 and 397-576 are said to respectively represent a homeobox domain and a homeodomain (HOX region).

NCBI # U81511 (SEQ ID NOs 241, 242, and 243) relate to a human HOXA-9A and HOXA-9B (HOXA-9) gene, alternatively spliced, complete cds. Nts 145-502, 4327-4894, and 5893-6131 are said to be exon (coding) sequences, with introns present at 503-5892 and 4895-5892. Alternative splicing events are said to account for the overlap.

t(8;16)(p11;p13)

Panagopoulos et al., Genes Chromosomes Cancer. 2000 August; 28(4):415-24, used RT-PCR analysis to identify MOZ-CBP and CBP-MOZ chimeric transcripts in acute myeloid leukemias with t(8; 16)(p11;p13) translocations.

NCBI # AJ251844 (SEQ ID NOs 244 and 245) relate to human partial mRNA for MOZ/CBP chimeric transcript type II. Nt 1-188 are said to derive from chromosome 8 and nts 189-415 from chromosome 16.

NCBI # AJ251845 (SEQ ID NOs 246 and 247) relate to a human partial mRNA for CBP/MOZ chimeric transcript. Nt 1-110 are said to derive from chromosome 16 and nts 111-229 from chromosome 8.

NCBI # AJ251843 (SEQ ID NOs 248 and 249) relate to human partial mRNA for MOZ/CBP chimeric transcript type I. Nt 1-188 are said to derive from chromosome 8 and nts 189-1128 from chromosome 16.

NCBI # U47742 (SEQ ID NOs 250 and 251) relate to human monocytic leukaemia zinc finger protein (MOZ) mRNA, complete cds.

NCBI # U85962 (SEQ ID NOs 252 and 253) relate to a human CREB-binding protein mRNA, complete cds. Nt 814-8147 are said to contain coding sequence and nts 819-1124 are said to encode a nuclear receptor binding domain.

t(9;12)(q34;p13)

Papadopoulos et al., Cancer Res. 1995 Jan. 1; 55(1):34-8, reported activation of ABL by fusion to an ets-related gene, TEL.

NCBI # Z35761 (SEQ ID NOs 254 and 255) relate to a human TEL/ABL fusion protein. Nt 1-463 are said to contain a partial TEL sequence and nt 464-549 are said to contain ABL sequence.

NCBI # Z36279 (SEQ ID NO 256) relates to human (9TX) breakpoint position DNA. The breakpoint position is said to reside at 567.568.

NCBI # Z36278 (SEQ ID NO 257) relates to human (boucher) breakpoint position DNA. The breakpoint position is said to reside at 567.568.

t(12;22)(p13;q13)

Buijs et al., Oncogene. 1995 Apr. 20; 10(8):1511-9, reported that a t(12;22) (p13;q11) event resulted in a myeloproliferative disorders characterized by the fusion of the ETS-like TEL gene on 12p13 to the MN1 gene on 22q11.

NCBI # X85024 (SEQ ID NOs 258 and 259) relate to a human mRNA for TEL-MN1 fusion gene (type II). Nt 22.23 is said to be the fusion site.

NCBI # X85026 (SEQ ID NOs 260 and 261) relate to a human mRNA for a TEL-MN1 fusion gene (type I). Nt 22.23 is said to be the fusion site.

NCBI # X85027 (SEQ ID NOs 262 and 263) relate to a human mRNA for a MN1-TEL fusion gene (type II). Nt 22.23 is said to be the fusion site.

NCBI # X85025 (SEQ ID NOs 264 and 265) relate to a human mRNA for a MN1-TEL fusion gene (type I). Nt 22.23 is said to be the fusion site.

del(5q)

Jaju et al., Blood 1999 Jul. 15; 94(2):773-80, reported a recurrent translocation, t(5;11)(q35;p15.5), associated with a del(5q) in childhood acute myeloid leukemia. Partial deletion of the long arm of chromosome 5, del(5q), is the cytogenetic hallmark of the 5q-syndrome, a distinct subtype of myelodysplastic syndrome-refractory anemia (MDS-RA). Deletions of 5q also occur in the full spectrum of other de novo and therapy-related MDS and acute myeloid leukemia (AML) types, most often in association with other chromosome abnormalities. However, the loss of genetic material from 5q is believed to be of primary importance in the pathogenesis of all del(5q) disorders.

Lindgren et al., Am J Hum Genet. 1992 May; 50(5):988-97, reported phenotypic, cytogenetic, and molecular studies of three patients with constitutional deletions of chromosome 5 in the region of the gene for familial adenomatous polyposis, APC, affiliated with colon cancer and polyps. High-resolution banding studies indicated that some deletions spans the region 5q21-q22.

Other potential deletion aberrations at the 5q locus include but are not limited to deletions at positions 5q13.3, corresponding to the RASA1 gene encoding the GAP RAS p21 protein activator 1 (GTPase activating protein), aberrancies of which are known to associate with basal cell carcinoma; 5q21, corresponding to the PST gene encoding PST1 Polysialyltransferase; 5q21-q22, corresponding to the APC gene, aberrancies of which correlate with colorectal cancer; 5q31, corresponding to the FACL6 gene encoding ACS2 Fatty-acid-Coenzyme A ligase, a long-chain 6 (long-chain acyl-CoA synthetase 2), aberrancies of which give rise to myelodysplastic syndrome and acute myelogenous leukemia; 5q31, encoding the GRAF GTPase regulator associated with the focal adhesion kinase, aberrancies of which give rise to juvenile myelomonocytic leukemia; 5q31.1, encoding IRF1, a MAR Interferon regulatory factor-1, aberrancies of which give rise to macrocytic anemiam myelodysplastic syndrome (preleukemic), acute myelogenous leukemia, gastric cancer, and nonsmall cell lung cancer; 5q33.2-q33.3, corresponding to CSF1R, FMS Colony-stimulating factor-1 receptor, aberranceis of which have been associated with oncogene FMS (McDonough feline sarcoma), and predisposition to myeloid malignancy; 5q35, encoding NPM1 Nucleophosmin 1 (nucleolar phosphoprotein B23, numatrin), aberrancies of which are known to associate with acute promyelocytic leukemia; 5q35.3, encoding gene FLT4, VEGFR3, encoding PCL fms-related tyrosine kinase-4 (vascular endothelial growth factor receptor, aberrancies of which contribute to hereditary lymphedema.

NCBI # NM_{—}002387 (SEQ ID NOs 266 and 267) relate to a human gene that is found mutated in colorectal cancers (MCC) mRNA. Nt 221-2710 are said to represent coding sequence. Allelic variation is said to exist at nt 2869 (c/t).

del(7q)

Schwartz et al., Cytogenet. Cell Genet. 51: 152-153 (1991) reported deletion mapping of plasminogen activator inhibitor, type I (PLANH1) and beta-glucuronidase (GUSB) in 7q21-q22. Wedemeyer et al., Genomics 46: 313-315 (1997) reported the proximity of the human HIP1 gene close to the elastin (ELN) locus on 7q11.23. Dridi et al., Am. J. Med. Genet. 87: 134-138 (1999), reported skin elastic fibers in Williams syndrome and Dutly et al., Am. J. Med. Genet. 87: 134-138 (1999), reported unequal interchromosomal rearrangements corresponding to deletions in these genes, and affiliated with Williams-Beuren syndrome. Naritomi et al., Hum. Genet. 80: 201-202 (1988), reported a microdeletion of the proximal long arm of chromosome 7 affiliated with Zellweger syndrome. Horiike et al., Leukemia. (1999) August; 13(8):1235-42, reported distinct genetic involvement of the TP53 gene in therapy-related leukemia and myelodysplasia, with chromosomal 7 losses and their possible relationship to replication error phenotype and the development of therapy-related AML/MDS. Wong et al., Cancer Genet Cytogenet. 1995 Jul. 1; 82(1):70-2, reported biclonal acute monoblastic leukemia associated with del(7q). Particular sites of interest include 7q11.23, encoding PTPN12, PTPG1 Protein tyrosine phosphatase, nonreceptor-type, known to associate with colon cancer; 7q21-q22, encoding PEX1, ZWS1 Peroxisome biogenesis factor-1, associate with Zellweger syndrome-1, neonatal adrenoleukodystrophy and infantile Refsum disease; 7q22-q31.1, encoding SLC26A3, DRA, CLD Solute carrier family 26 (sulfate transporter), member 3, associated with colon cancer; 7q31-q32 SMOH, SMO Smoothened, Drosophila, homolog of 601500, associated with sporadic basal cell carcinoma.

del(20q)

A deletion in the long arm of chromosome 20 is a recurring abnormality in malignant myeloid disorders. Its occurrence suggests that the loss of genetic material on 20q provides a proliferative advantage to myeloid cells, possibly through the loss of a tumor-suppressor gene. Roulston et al., Blood 82: 3424-3429 (1993), examined a series of patients with the del(20q) using fluorescence in situ hybridization with unique sequence probes that map along the length of 20q and delineated a segment that is deleted in 95% of all patients they examined (18 of 19). In addition, they showed that the deletions are interstitial rather than terminal. The region of deletion extended from 20q11.2 to 20q12 and was flanked by RPN2 (180490) proximally and D20S17 distally. The SRC (190090) and ADA (102700) genes were found to be located within the commonly deleted segment.

Stoffel et al. (1996) generated a YAC contig map of 20q11.2-q13.1 in a region spanning about 18 Mb and representing about 40% of the physical length of 20q. The map contains the chromosomal regions deleted in MODY1 (125850) and in myeloid leukemia. Using this physical map, they refined the location of a myeloid tumor suppressor-related gene to an 18-cM interval (approximately 13 Mb) between RPN2 and D20S17.

Stoffel et al., Proc. Nat. Acad. Sci. 93: 3937-3941 (1996), correlated the occurrence of del(20q) in a broad spectrum of myeloid disorders, suggesting that the loss of genetic material on 20q could provide a proliferative advantage to myeloid cells, possibly through the loss of a tumor-suppressor gene. Stoffel et al. examined a series of patients with the del(20q) using fluorescence in situ hybridization (FISH) with unique sequence probes that map along the length of 20q, delineated a segment that is deleted in 95% of all patients examined (18 of 19), and showed that the deletions are interstitial rather than terminal. This region of deletion extends from 20q11.2 to q12, and is flanked by the RPN2 (proximal) and D20S17 loci (distal). The SRC and ADA genes are located within the commonly deleted segment.

t(11q23)

Shiah et al., Leukemia, (2002) 16(2):196-202, reported clinical and biological implications of partial tandem duplication of the MLL gene in acute myeloid leukemia without chromosomal abnormalities at 11q23. The clinical and biological features of acute myeloid leukemia (AML) with 11q23/MLL translocations are well known, but the characteristics of AML with partial tandem duplication of the MLL gene have not been explored comprehensively. Sheah et al analyzed MLL duplication in 81 AML patients without chromosomal abnormalities at 11q23, using Southern blotting, genomic DNA polymerase chain reaction (PCR), reverse-transcription PCR and complementary DNA sequencing. Nine patients showed partial tandem duplication of the MLL gene, including eight (12%) of the 68 with normal karyotype. Seven patients showed fusion of exon 6/exon 2 (e6/e2), one, combination of differentially spliced transcripts e7/e2 and e6/e2, and the remaining one, combination of e8/e2 and e7/e2. Among the patients with normal karyotype, children aged 1 to 15 showed a trend to higher frequency of MLL duplication than other patients (2/5 or 40% vs 6/62 or 10%, P=0.102). The patients with tandem duplication of the MLL gene had a significantly higher incidence of CD11b expression on leukemic cells than did those without in the subgroup of patients with normal karyotype (75% vs 28%, P=0.017). There were no significant differences in the expression of lymphoid antigens or other myeloid antigens between the two groups of patients. In adults, the patients with MLL duplication had a shorter median survival time than those without (4.5 months vs 12 months, P=0.036). In conclusion, partial tandem duplication of the MLL gene is associated with increased expression of CD11b on leukemic blasts and implicates poor prognosis in adult AML patients. The higher frequency of MLL duplication in children older than 1 year, than in other age groups, needs to be confirmed by further studies.

Ono et al., Cancer Res. 2002 Jan. 15; 62(2):333-7, reported that SEPTIN6, a human homologue to mouse Septin6, is fused to MLL in infant acute myeloid leukemia with complex chromosomal abnormalities involving 11q23 and Xq24.

Borkhardt et al., Genes Chromosomes Cancer. 2001 September; 32(1):82-8, reported an ins(X;11)(q24;q23) that fuses the MLL and the Septin 6/KIAA0128 gene in an infant with AML-M2.

Luo et al., Mol Cell Biol. 2001 August; 21(16):5678-87, reported that ELL-associated factor 1 interaction domain is essential for MLL-ELL-induced leukemogenesis.

Kuwada et al., Cancer Res. 2001 Mar. 15; 61(6):2665-9, reported a t(11;14)(q23;q24) that generates an MLL-human gephyrin fusion gene along with a de facto truncated MLL in acute monoblastic leukemia.

Garcia-Cuellar et al., Oncogene. 2000 Mar. 30; 19(14):1744-51, reported that ENL, the MLL fusion partner in t(11; 19), binds to the c-Abl interactor protein 1 (ABI1) that is fused to MLL in t(10; 11)+.

Akao et al., Genes Chromosomes Cancer. 2000 April; 27(4):412-7, reported an analysis of the rearranged genome and chimeric mRNAs caused by a t(6;11)(q27;q23) chromosome translocation involving MLL in an infant acute monocytic leukemia.

Hayashi et al., Cancer Res. 2000 Feb. 15; 60(4):1139-45, reported a leukemic cell line, SN-1, associated with a t(11;16)(q23;p13.

So et al., Cancer Genet Cytogenet. 2000 February; 117(1):24-7, analysed MLL-derived transcripts in an infant acute monocytic leukemia having a complex translocation (1;11;4)(q21;q23;p16).

Kourlas et al., Proc Natl Acad Sci USA. 2000 Feb. 29; 97(5):2145-50, identified a gene at 11q23 encoding a guanine nucleotide exchange factor that fuses with MLL in acute myeloid leukemia.

Taki et al., Proc Natl Acad Sci USA. 1999 Dec. 7; 96(25):14535-40, reported that AF5q31, an AF4-related gene, is fused to MLL in infant acute lymphoblastic leukemia with an ins(5;11)(q31;q13q23).

Taki et al., Cancer Res. 1999 Sep. 1; 59(17):4261-5, reported that AF17q25, a putative septin family gene, fuses with the MLL gene in acute myeloid leukemia associated with a t(11;17)(q23;q25).

Busson-Le Coniat et al., Leukemia. 1999 February; 13(2):302-6, reported MLL-AF1q fusion resulting from t(1;11) in an acute leukemia.

Slany et al., Mol Cell Biol. 1998 January; 18(1):122-9, reported on the oncogenic capacity of HRX-ENL that requires the transcriptional transactivation activity of ENL and the DNA binding motifs of HRX.

Other articles of interest include, Super et al., Genes Chromosomes Cancer. 1997 October; 20(2): 185-95, Identification of complex genomic breakpoint junctions in the t(9;11) MLL-AF9fusion gene in acute leukemia; Taki et al., Blood. 1997 Jun. 1; 89(11):3945-50, The t(11;16)(q23,p13) translocation in myelodysplastic syndrome fuses the MLL gene to the CBP gene; Taki Tet al., Fusion of the MLL gene with two different genes, AF-6 and AF-5alpha, by a complex translocation involving chromosomes 5, 6, 8 and 11 in infant leukemia, Oncogene. 1996 Nov. 21; 13(10):2121-30. Tanabe et al., AF10 is split by MLL and HEAB, a human homolog to a putative Caenorhabditis elegans ATP/GTP-binding protein in an invins(10;11)(p12;q23q12), Blood. 1996 Nov. 1; 88(9):3535-45; Ma et al., LAF-4 encodes a lymphoid nuclear protein with transactivation potential that is homologous to AF-4, the gene fused to MLL in t(4;11) leukemias, Blood. 1996 Jan. 15; 87(2):734-45; Prasad et al., Domains with transcriptional regulatory activity within the ALL1 and AF4 proteins involved in acute leukemia, Proc Natl Acad Sci USA. 1995 Dec. 19; 92(26):12160-4. Baffa et al., Involvement of the ALL-1 gene in a solid tumor, Proc Natl Acad Sci USA. 1995 May 23; 92(11):4922; Mitani, Cloning of several species of MLL/MEN chimeric cDNAs in myeloid leukemia with t(11;19)(q23,p13.1) translocation, Blood. 1995 Apr. 15; 85(8):2017-24; Tse et al., A novel gene, AF1q, fused to MLL in t(1;11) (q21;q23), is specifically expressed in leukemic and immature hematopoietic cells, Blood. 1995 Feb. 1; 85(3):650-6; Chen et al., Acute promyelocytic leukemia: from clinic to molecular biology, Stem Cells. 1995 January; 13(1):22-31. Review; Rubnitz et al., ENL, the gene fused with HRX in t(11;19) leukemias, encodes a nuclear protein with transcriptional activation potential in lymphoid and myeloid cells, Blood. 1994 Sep. 15; 84(6):1747-52; Prasad et al., Leucine-zipper dimerization motif encoded by the AF17 gene fused to ALL-1 (MLL) in acute leukemia, Proc Natl Acad Sci USA. 1994 Aug. 16; 91(17):8107-11; Meerabux et al., Molecular cloning of a novel 11q23 breakpoint associated with non-Hodgkin's lymphoma, Oncogene. 1994 March; 9(3):893-8; Gauwerky et al., Chromosomal translocations in leukaemia, Semin Cancer Biol. 1993 December; 4(6):333-40. Review; Hunger et al., HRX involvement in de novo and secondary leukemias with diverse chromosome 11q23 abnormalities, Blood. 1993 Jun. 15; 81(12):3197-203; Morrissey et al., A serine/proline-rich protein is fused to HRX in t(4;11) acute leukemias, Blood. 1993 Mar. 1; 81(5):1124-31; Tkachuk et al., Involvement of a homolog of Drosophila trithorax by 11q23 chromosomal translocations in acute leukemias, Cell. 1992 Nov. 13; 71(4):691-700.

t(5;12)(q31;p13)

Yagasaki et al. described a fusion of LACS to a TEL/ETV6 gene in an acute myeloblastic leukemia case having a t(5;12) chromosomal translocation. The human mRNA fusion sequence may be found in NCBI # AF102845 (SEQ ID NO 268). Nt 1-40 are said to derive from the TEL gene on chromosome 12 and nt 41-1172 are said to derive from the LACS gene on chromosome 5.

t(1;12)(q25;p13)

Cazzaniga et al., Blood 94: 4370-4373 (1999), reported an instance of the tyrosine kinase Abl-related gene ARG fused to ETV6 in an AML-M4Eo patient having a t(1;12)(q25;p13) translocation, and cloned reciprocal chimeric transcripts associated with the event. The ETV6/TEL gene is rearranged in most patients with 12p13 translocations fused to a number of different partners. One of the chimeric proteins consisted of the helix-loop-helix oligomerization domain of ETV6 and the SH2, SH3, and protein tyrosine kinase domains of ABL2. The reciprocal transcript ABL2-ETV6 was also detected in the patient's RNA by RT-PCR, although at a lower expression level.

t(12;15)(p13;q25)

Wai et al., Oncogene. 2000 Feb. 17; 19(7):906-15, reported an ETV6-NTRK3 gene fusion associated with such translocation.

Eguchi et al., Blood. 1999 Feb. 15; 93(4):1355-63, reported a similar fusion of ETV6 to neurotrophin-3 receptor TRKC in acute myeloid leukemia with t(12;15)(p13;q25).

Knezevich et al., Nat. Genet. 1998 February; 18(2):184-7; reported an ETV6-NTRK3 gene fusion in congenital fibrosarcoma.

NCBI # AF125808 (SEQ ID NOs 269 and 270) relate to a human ETS related protein-neurotrophic receptor tyrosine kinase fusion protein (ETV6-NTRK3 fusion) mRNA, partial cds. Nt 12-64 are said to derive from chromosome 12 and nt 65-980 from chromosome 15.

NCBI # AF041811 (SEQ ID NOs 271 and 272) relate to a human ETS related protein-growth factor receptor tyrosine kinase fusion proteins (ETV6-NTRK3 fusion) mRNA, partial cds. Nt 1-336 are said to derive from chromosome 12 and nt 337-1403 from chromosome 15.

t(1;12)(q21;p13)

Salomon-Nguyen et al., Proc Natl Acad Sci USA. (2000) 97(12):6757-62, reported a t(1;12)(q21;p13) translocation observed in a case of acute myeloblastic leukemia (AML-M2). At the protein level, the untranslocated TEL copy and, as a result of the t(1;12) translocation, a fusion protein containing the amino-terminal part of TEL and essentially all of the ARNT gene (126110), were expressed. The TEL/ETV6 gene is located at 12p 13 and encodes a member of the ETS family of transcription factors. Translocated ETS leukemia (TEL) is frequently involved in chromosomal translocations in human malignancies, usually resulting in the expression of fusion proteins between the amino-terminal part of TEL and either unrelated transcription factors or protein tyrosine kinases. ARNT (aryl hydrocarbon receptor nuclear translocator) belongs to a subfamily of the “basic region helix-loop-helix” (bHLH) protein that shares an additional region of similarity called the PAS (Per, ARNT, SIM) domain. ARNT is the central partner of several heterodimeric transcription factors, including those containing the aryl hydrocarbon (dioxin) receptor (AhR) and the hypoxia-inducible factor 1 alpha (HIF1 alpha). Interference with the activity of AhR or HIF1 alpha may contribute to leukemogenesis.

2. Mutant Protein or Cellular Protein Isoforms

The second group of target proteins are mutants or isoforms (e.g. splice variants) of normal cellular proteins (usually the products of tumor suppressor genes) that, due to their mutant nature, exhibit a heightened dependence on HSP90 chaperone functions or else increased senstivity, i.e., instability, due to HSP90 inhibitors. The mutant or isoform proteins either (a) have become overtly oncogenic (a “dominant-positive” (DP) effect), or (b) exert a “dominant-negative” (DN) effect on their normal counterpart, thus preventing the normal protein's tumor suppressor activity, and resulting in a net oncogenic effect. The examples are largely illustratd with respect to human sequences, although the person of ordinary skill will appreciate that homologs in other organisms are likewise included within the purview of the invention.

a. v-src

One such example of a mutant or isoform protein is human v-src (NCBI #s NM_{—}005417; SEQ ID NOs 273 and 274), which counterpart, c-src (NCBI # XM_{—}044659 (SEQ ID NOs 275 and 276), corresponds to the normal cellular gene product. As described above, proteins with a heightened dependence on HSP90 can be identified by their enhanced sensitivity to degradation induced by HSP90 inhibitors, such as the ansamycin antiobiotic geldanamycin. Ansamycins and other HSP90 inhibitors were originally isolated on the basis of their ability to revert v-src transformed fibroblasts (Uehara, Y. et al., 1985, Supra, 76: 672-675) and this reversal was correlated with the functional inactivation of the v-src protein (Uehara, Y. et al., 1986, Mol. Cell. Biol., 6: 2198-2206). This effect was subsequently reported to be caused by the ubiquitin/proteosome-dependent degradation of the transforming v-src protein as a result of inhibition of HSP90 function by geldanamycin (Whitesell, L., et al., 1994, supra). Finally, a recent study compared the rate and potency of degradation of v-src and c-src proteins after treatment of Rous sarcoma virus-transformed 3T3 fibroblasts with the ansamycin geldanamycin. In this study, the oncogenic mutant v-src protein was almost 100% degraded within 6 hours (An, W et al, 2000, supra, see FIG. 2), whereas the normal cellular counterpart, c-src, was largely unaffected even after 20 hours of the same treatment (An, W et al, 2000, supra, see FIG. 4).

HSP90 inhibitors can selectively induce degradation of a wide range of mutated or otherwise aberrant proteins that cause or exacerbate a disease, and that have an apparent heightened dependence on HSP90.

b. RET

An example of a dominant proto-oncogene encoding a signaling protein that is mutated in certain human cancers giving rise to constitutively active structurally abnormal cellular proteins is the RET proto-oncogene (NCBI # P07949; SEQ ID NO 277) in multiple endocrine neoplasia Type 2 (MEN-2). RET encodes a receptor tyrosine kinase whose ligand is presently unidentified (Kolibaba, K, et al, 1997, Supra). The germline mutations found in MEN-2A patients (Cys634→Arg/Tyr, similar mutations at Cys609, 611, 618 and 620) alter the tertiary structure of the protein resulting in homodimerization and activation of the kinase domain. The commonly observed mutation in MEN-2B, Met918→Thr, alters the kinase domain structure, causing activation directly. Both of these pathways involve alterations in protein conformation, which again implicates HSP90 and underscores the broad utility of the invention.

c. p53

Another example of a mutant, oncogenic variant group of a normal cellular protein is tumor suppressor antigen p53. The wild-type protein and mRNA sequences for p53 are found in NCBI accession # M14695 (SEQ ID NOs 278 and 279). However, numerous mutations in p53 are known to occur and represent the most common molecular genetic defects found in human cancers (Harris, C et al, 1993, N. Engl. J. Med. 329:1318-1327). Amutantp53 protein was reportedly degraded in cells following treatment with geldanamycin, but wild type p53 exhibited no such, or only minimal, degradation (Blagosklonny, M et al, 1995, Oncogene, 11:933-939). Unlike the situation described above for v-src, most p53 mutations are “loss of function” effects, i.e., the mutation results in the inability of the protein to perform one or more of its normal functions. Thus, in a tumor cell that has an intact p53 allele and a loss of function mutant allele, simply causing the mutant form to be degraded will not change cellular behavior. However, if the mutant protein by some mechanism inhibits the action of its coexpressed normal counterpart inside tumor cells, then degrading it will affect cellular behaviour.

This “dominant-negative” (DN) effect has been shown to occur in cells harboring certain p53 mutants, and by several different mechanisms. For example, a mutant may afford tighter DNA binding without transactivation (Chene, P, et al, 1999, Int. J. Cancer. 82:17-22). This type of p53 mutant does not exhibit “classical” DN activity unless the mutation confers an increased affinity for DNA, because the mutant stoichiometrically competes with the wild type (WT) protein for binding to DNA. Another example is inhibition of tetramerization by incorporation of one or more mutant p53s into a complex with WT proteins (Deb, D et al, 1999, Int. J. Oncol. 15:413-422, Rollenhagen, C et al, 1998, Int. J Cancer 78:372-376). Yet a third example concerns “prion-like” activity, in which a mutant protein forces a WT protein into a mutant conformation that then impairs its ability to bind to DNA and/or transactivate p53 target genes (Chene, P, 1998, J. Mol. Biol. 281:205-209)

Increased stability of mutants relative to WT proteins causes them to accumulate and override normal p53 biology. This is counterintuitive given the fact that p53 has a built-in negative feedback loop on its own transcription (via induction of the mdm-2 protein, which subsequently targets p53 for degradation). If the increased stability of a given mutant were due solely to failure to transactivate mdm-2, then accumulation of the mutant would not occur in the presence of a WT allele (Blagosklonny, M, 2000, FASEB J 14:1901-1907) because this protein would initiate negative feedback mechanisms that would be expected to act on both WT and mutant p53.

On the other hand, an independent mechanism favoring mutant accumulation (e.g. protection by association with HSP90 (Smith, D, et al, 1998, supra; Sepehrnia, B, et al, 1996, J. Biol. Chem. 271:15084-15090) would permit a “recessive” mutant to become in sufficient excess of the transactivating form to result in progressive inhibition of the negative feedback pathways. In this situation, the mutant would have a net DN effect due to progressive accumulation of a stoichiometric antagonist, and selective degradation of that mutant by inhibition of HSP90 activity would be expected to restore normal p53 function. Thus, in most or all cases, a DN phenotype produced by mutant p5³ is secondary to the activity of HSP90 and inhibition of HSP90 function with 17-AAG or other HSP90 ATP binding site antagonists would prevent the expression of the DN phenotype and so rescue normal p53 function.

i. Dominant Negative p53 Mutants

A list of exemplary p53 mutations, including examples of structurally-abnormal proteins, dominant-negative proteins, prion-like proteins, and mutants with various combinations of these properties, follows:

Chene et al, 1999, Int. J. Cancer. 82:17-22; Y236delta (deletion of codon 236) resulted in a conformationally altered & dominant-negative phenotype.

Preuss et al, 2000, Int. J. Cancer 88:162-171); C174Y (Cys→Tyr) (rat) is dominant-negative, non-transactivating. The same mutation at position 176 is predicted to have a similar effect in humans, as the respective homologs have close correlative structural similarities at these positions.

Srivastava et al, 1993, Oncogene 8:2449-2456); M133T (Met→Thr), G245D (Gly→Asp), and E258K (Glu→Lys) all display conformationally altered, dominant-negative, prion-like displaying activity, in that co-incubation with WT p53 converts it into the mutated conformation.

Deb et al, 1999, Int. J. Oncol. 15:413-422); 1-293delta (deletion of codons 1-293) exhibited dominant negative DNA binding characteristics without transactivating activity.

Frebourg et al, 1992, Proc. Natl. Acad. Sci. 89:6413-6417; G245C (Gly→Cys), R248W (Arg→Trp), E258K (Glu→Lys), and R282W (Arg→Try) all independently display conformationally altered, dominant-negative activity.

Brachmarn et al, 1996, Proc. Natl. Acad. Sci. 93:4091-4095; novel yeast assay used to identify dominant-negative p53 mutants that have also been found in human tumors, specifically implicating codons 132, 135, 151, 158, 176, 179, 236, 241, 242, 244, 245, 246, 248, 257, 265, 273, 277, 278, 279, 280, and 281. Of particular interest because they exhibited the greatest dominant-negative activity were mutants at codons 241, 242, 244, 245, 246, 248, 277, 278, 279, 280, and 281.

Blagosklonny et al, 1995, Oncogene 11:933-939); p53s mutated at the following codons exhibited disrupted conformations were dominant negative, and sensitive to geldanamycin: R175H (Arg→His), 194, 213, 223, 248, 274, R280K (Arg→Lys).

Aurelio et al, 2000, Mol. Cell. Biol. 20:770-778; without identifying conformational status, the following mutants were identified as dominant-negative for transactivation of apoptotic signals (Bax), but not growth arrest signals (p21^{WAF}): V143A (Val→Ala), R175H (Arg→His), G245C (Gly→Cys), R248W (Arg→Trp), R273H (Arg→His), K305M (Lys→Met), G325V (Gly→Val).

Marutani et al, 1999, Cancer Res. 59:4765-4769; yeast-based transdominance assay used to identify dominant-negative mutations at 16 codons: R156H (Arg→His), R175H (Arg→His), P177S (Pro→Ser), H178P (His→Pro), H179R (His→Arg), R181P (Arg→Pro), 238-9delta (deletion of codons 238 & 239), G245S (Gly→Ser), G245D (Gly→Asp), M246R (Met→Arg), R248Q (Arg→Gln), R249S (Arg→Ser), R273H (Arg→His), R273c (Arg→Cys), R273L (Arg→Leu), D281Y (Asp→Tyr).

Ii. Dominant Positive p53 Mutants

In addition to dominant-negative mutations, some p53 mutations actually transactivate inappropriate gene expression, contributing to oncogenesis; i.e. a positive tumor promoting effect. See Park et al, 1994, Oncogene 9:1899-1906. This type of mutation is particularly suited to the approach embodied in the present invention because, unlike in the dominant-negative situation, the presence or absence of a normal allele of the tumor suppressor gene is irrelevant to the therapeutic utility of the HSP90 inhibitor. In other words, because the mutant p53 itself contributes to the malignant process, destruction of the mutant protein by inhibition of HSP90 is expected to have direct therapeutic value. A good example is C176Y (Cys→Tyr), as reported by Preuss, U et al, 2000, Int. J. Cancer 88:162-171. This mutant induces rather than represses the cellular fos promoter, resulting in activation of oncogenic signaling pathways. The biology of “dominant-positive” p53 mutants is reviewed in van Oijen et al, 2000, Clin. Cancer Res. 6:2138-2145. Other examples of mutations of p53 that give rise to tumorigenic phenotypes include, but are not limited to, Phe-132, Val-135, Ala-143, His-175, His-179, Trp-248, Ser-249, Leu-273, His-273 and Gly-281. Of particular interest, because these mutant proteins have been shown to be disrupted conformationally, are Ala-143, His-175, His-179, and Gly-281 (van Oijen, M, et al, 2000, supra). Particular subsets of the above list of tumor-promoting mutants have been shown to exert their oncogenic effects via transactivation of one or more of the growth promoting genes bFGF, IGF-1, EGF-R, and c-myc. Alternatively or conjunctively, some gain-of-function mutants, including Ala-143, His-175, Trp-248, Ser-249, His-273, and Gly-281, contribute to tumor resistance to chemotherapeutic drugs by transactivating the MDR gene.

As described above, in the case of this type of mutant, in heterozygous cells, selective degradation of that mutant by inhibition of HSP90 activity will restore normal p53 function. Furthermore, in cases of loss of heterozygosity (LOH), where the tumor has progressed further and the second, normal p53 allele has become mutated or lost, selective degradation of the mutated protein by inhibition of HSP90 chaperoning will result in a therapeutic effect. In this case the p53 mutant is behaving as an oncoprotein, as in the bcr-abl and v-src examples described above.

D. Other Tumor Suppressor Variant Proteins

In addition to p53 itself, additional members of the p53 family of tumor suppressor proteins have also been implicated in human cancer progression. Although p53 itself is a fairly ubiquitous protein, other family members have more restricted tissue distributions. In particular tissues and tumors derived therefrom, closely related non-p53 proteins serve the same role as p53 itself. In these tumors, a truncated variant, termed deltaN, predominates over the full-length form. The truncated and/or deletent isoform is able to compete with the full length form for DNA binding, but does not itself have any transactivating activity. Thus, the deltaN form inhibits the tumor suppressor activity of the full length form, so that if the variant is degraded as a result of inhibition of HSP90 activity, an antitumor effect or drug-sensitizing effect will result. The deltaN isoform will have a heightened dependence on HSP90.

The following three examples concern the specific tumor suppressor proteins p51, p63, and p73. p51 and p63 are each produced from a common 15 exon gene, p73L/p63/p51/p40/KET, and all three proteins exhibit various isoforms, including deltaN isoforms that lack N-terminal transactivation (TA) domains and which are implicated in various carcinomas treatable according to methods of the invention. The many isotypes possible for these gene products are attributable, at least in part, to complex alternative splicing events and, in the case of p63, multiple promoters. For each, it is understood that isoforms may exist and specific isoform expression patterns may vary as between different tissue types, and as between normal versus carcinomic or neoplastic tissues.

i. deltaN p51

Osada et al. described the cloning and functional analysis of human p51, which structurally and functionally resembles p53. Nature Med. 4: 839-843 (1998). Two major splicing variant gene products have been detected in normal cells, p51A and p51B. p51A (aka TAp63gamma; NCBI #s AB016072 (SEQ ID NOs 280 and 281) is a 448-amino-acid protein with a molecular weight of 50.9 kDa; and p51B (aka TAp63alpha; AB016073 (SEQ ID NOs 282 and 283) is a 641-amino-acid protein with a molecular weight of 71.9-kDa. Other encoded isoforms have also been observed, including, e.g., those denoted in the following list: p51 delta (NCBI # AF116771 (SEQ ID NOs 284 and 285), delNdelta (NCBI # AAF43493 (SEQ ID NOs 286 and 287), delNbeta (NCBI # AAF43492 (SEQ ID NOs. 288 and 289), delNalpha (NCBI # AAF43491 (SEQ ID NOs. 290 and 291), delNgamma (NCBI # AAF43490; SEQ ID NOs 292 and 293), TAp63delta (NCBI # AAF43489; SEQ ID NOs 294 and 295), TAp63beta (NCBI # AAF43488 (SEQ ID NOs 296 and 297), TAp63alpha (NCBI #AAF43487 (SEQ ID NOs 298 and 299), and TAp63gamma (NCBI # AAF43486 (SEQ ID NOs 300 and 301). The TA isoforms contain a transactivation domain (encoded by exon 3′) for transactivating p53; the deltaN forms do not. The absence of the TA domain is thought to render those particular isoforms nonfunctional, thereby contributing to carcinoma etiology at least when those isoforms are expressed in abnormally high amounts. Normal expression patterns of the various isotypes is known to vary as between different tissue types. In lung cancer specimens, for example, multiple deltaN (“TA-less”) forms of the p51 protein were found to be overexpressed in 34 of 44 lung cancer specimens analysed (77%). (Tani, M et al, 1999, Neoplasia 1:71-79).

ii. deltaN p63

In certain bladder and nasopharyngeal carcinomas, various isoforms of the p53 family member p63 are expressed, and one or more of the deltaN forms, e.g., deltaN p63beta (NCBI #AF075433; SEQ ID NOs 302 and 303), deltaN p63gamma (NCBI #AF075429; SEQ ID NOs 304 and 305), and deltaN p63 alpha (NCBI #AF075431 (SEQ ID NOs 306 and 307) predominate and dominantly inhibit the transactivating activity of the full length TA-containing forms. (Park, B et al, 2000, Cancer Res. 60:3370-3374). The TA-containing isoforms are TA p63 beta (NCBI #AF075432; SEQ ID NOs 308 and 309) and TA p63 alpha (NCBI #AF075430; SEQ ID NOs 310 and 311). In nasopharyngeal carcinoma, the deltaN isoform predominance is even more pronounced (Crook, T et al, 2000, Oncogene 19:3439-3444). The p63 protein is also important in UV-B-induced skin cancer. Overexpression of the deltaN isoform of p63 in transgenic mouse epidermis was found to block apoptosis induced by WT p53 in response to UV-B irradiation (Liefer, K, et al, 2000, Cancer Res. 60:4016-4020). Mutations in the p63 gene have also been reported in epidermal carcinomas. See, e.g., Osada et al, 1998, Nat. Med. 4:839-843 and NCBI #NM003722 (SEQ ID NOs 312 and 313).

iii. deltaN p73

The p73 protein is important in ovarian carcinoma—when compared to primary cultures of normal ovarian epithelial cells, 57% of ovarian carcinoma cell lines, 71% of invasive tumors and 92% of borderline tumor tissues were found to express elevated levels of deltaN p73 (Ng, S et al, 2000, Oncogene 19:1885-1890). Full-length p73 and isoforms thereof are displayed in NCBI # Y11416 (SEQ ID NOs 314, 315, 316, and 317), along with splice and allelic variations, including splice variations responsible for the deltaN isoform.

Applicants expect that all of the foregoing truncated p53 family members are structurally unstable, dependent on HSP90 and/or exhibit increased sensitivity to HSP90 inhibitors relative to their wild-type counterparts. Applicants further anticipate that other isomeric/aberrant forms of proteins may exhibit similar behavior(s).

The methods of the present invention may be used on mammals, preferably humans, either alone or in combination with other therapies or methods useful for treating a particular cell proliferative disorder or viral infection.

The use of the present invention is facilitated by first identifying whether the cell proliferation disorder or viral infection is accompanied by cells which contain expression of a fusion oncoprotein or a mutated cellular protein with heightened dependence on HSP90 (or a fusion protein or mutant protein that, by one skilled in the art, would be predicted to have heightened dependence on HSP90). Once such disorders are identified, patients suffering from such a disorder can be identified by analysis of their symptoms by procedures well known to medical doctors. Such patients are treated as described herein.

3. Representative Assays for Diagnosing Proliferative Disorders

Many different types of methods are known in the art that can be used to diagnose a proliferative disorder characterized by an aberrant protein, e.g., those that involve determining protein concentrations and measuring or predicting the level of proteins within cells, tissues, and fluid samples. Indirect techniques include nucleic acid hybridization and amplification using, e.g., polymerase chain reaction (PCR). These techniques are known to the person of skill and are discussed, e.g., in Sambrook, Fritsch & Maniatis, Molecular Cloning: A Laboratory Manual, Second Edition (1989) Cold Spring Harbor Laboratory Press, Cold Spring Harbor, N.Y., Ausubel, et al., Current Protocols in Molecular Biology, John Wiley & Sons, NY, 1994. Because the nucleic acid sequence is known, and because the aberrant proteins have a foundational basis in the nucleic acid sequence, the specific sequences found for aberrant proteins can also be used to generate primers and probes that span the novel junction (in the case of fusion proteins), e.g., using RT-PCR and other procedures. For non-fusion proteins, as well as fusion proteins, stringent hybridization and/or PCR can be used diagnostically.

Polyclonal or monoclonal antibodies can also be generated based on the specific sequence of the aberrant protein (in the case of fusion proteins, preferably the novel amino acid junction itself) using routine techniques. See Harlow et al., Antibodies: A Laboratory Manual, 2nd Ed; Cold Spring Harbor Laboratory Press, Cold Spring Harbor, N.Y. (1988).

Examples of diagnostic methods of that can be used with the invention include those reviewed in Slominski, A et al, 1999, Arch. Patlol. Lab. Med. 123:1246-1259, O'Connor et al, 1999, Leuk. Lymphoma 33:53-63, and Scarpa, A et al, 1997, Leuk. Lymphoma 26 Suppl. 1:77-82. A further list of methods that is intended to be exemplary but not to limit the scope of the invention, follows.

O'Connor et al, 1997, Br. J. Haematol. 99:597-604 described that the t(15;17)(q22:q21) translocation found in APL produces a PML-RAR fusion protein that can be specifically detected with the SE10 Mab by fluorescence activated cell sorting (FACS).

Le et al, 1998, Eur. J. Haematol. 60:217-225 reported that the AML-ETO fusion protein that arises in t(8;21) AML can be identified in tumor cells with ETO-specific polyclonal antibodies using western blotting. The normal ETO protein (70 kD) can be distinguished from the AML-ETO fusion protein (94 kD) on the basis of their differing mobilities in the gel.

Viswanatha et al, 1998, Blood 91:1882-1890 found that the CBFB-SMMHC fusion protein present in Inv(16)(p13q32) and t(16:16)(p13;q32) AML can be specifically detected with a polyclonal antibody specific for a junctional epitope using FACS of permeabilized cells.

In the case of dominantly-acting mutant proteins, such as mutant RET or gain-of-function mutants of p53, the presence of the specific point mutations known to give rise to the dominant mutant may be identified by the molecular genetic techniques listed above in reference to fusion proteins. Numerous reviews of germline and acquired p53 mutations detected in human cancers have been published (see, e.g., Hainuit, P, et al, 2000, Adv. Cancer Res. 77:81-137).

In the case of dominant-negative p53 mutations, several other diagnostic criteria may be employed to identify patients susceptible of treatment with the current invention. First, molecular genetic methodologies such as Southern Blotting or PCR can be used to detect the presence of a specific point mutation known to give rise to a dominant-negative version of p53. Similarly, FISH may be employed to detect specific point mutations known to confer conformational changes and/or dominant-negative activity (Villadsen R et al, 2000, Cancer Genet. Cytogenet. 116:28-34). Other methods include allele-specific PCR (AS-PCR) and chromosome flow cytometry (Villadsen et al, Supra).

Alternatively, if the mutation in question has not previously been shown to generate a dominant-negative p53 mutant, a cell-based transdominance assay may be used to determine the phenotype (Frebourg, T et al, 1992, Proc. Natl. Acad. Sci. 89:6413-6417). In this assay, p53-null SAOS-2 cells are co-transfected with WT p53 and the test mutant. The normal p53 protein causes the cells to undergo apoptosis, from which fate they can be rescued by a p53 mutant that has a dominant negative activity. In these cases, further genetic analyses may be performed to confirm the presence of an intact non-mutant allele. In addition, antibodies have been raised that distinguish between p53 proteins with normal versus mutant conformation. These latter p53s have a heightened dependence upon HSP90, and so fall within the scope of the present invention. Specifically, PAb240, from (Oncogene Sciences, Inc.) OSI, is mutant conformation-specific. The corresponding antibody specific for WT is PAb1620, also for OSI (Chene, P, et al, 1999, supra).

In the case of cell proliferative disorders arising due to unwanted proliferation of non-cancer cells, the level of the fusion protein or mutated cellular protein is compared to that level occurring in the general population (e.g., the average level occurring in the general population of people or animals excluding those people or animals suffering from a cell proliferative disorder). If the unwanted cell proliferation disorder is characterized by an abnormal level of a fusion protein than occurrs in a normal population, or by the presence of a mutated cellular protein, such as p53, then the disorder is a candidate for treatment using the methods described herein. In a preferred example, the mutated protein is p53 and the proliferative disorder is rheumatoid arthritis. In a particularly preferred example, the p53 mutations may include, but are not limited to, N239S (Asn→Ser), C176R (Cys-Arg) and R213* (Arg→stop) and the mutant forms exert apparent dominant-negative activity over the wild-type protein. (Han, Z et al, 1999, Arthritis Rheum. 42:1088-1092).

4. Preparation and Administration of Pharmaceutical Compositions

Geldanamycin may be prepared according to U.S. Pat. No. 3,595,955 using the subculture of Streptomyces hygroscopicus that is on deposit with the U.S. Department of Agriculture, Northern Utilization and Research Division, Agricultural Research, Peoria, Ill., USA, accession number NRRL 3602. It is also available from Sigma/Aldrich Chemical Co., St. Louis, Mo., USA. Numerous derivatives of this compound, including herbimycin A, macbecin, and 17-AAG may be fashioned as specified in U.S. Pat. Nos. 4,261,989, 5,387,584, and 5,932,566, or according to standard techniques known in the art. Other useful ansamycin derivatives appear in Applicants' co-pending and commonly owned provisonal application entitled, “Ansamycins Having Improved Pharmacological and Biological Properties,” filed Feb. 8, 2002, Serial Number to be determined, and herein incorporated by reference in its entirety.

Those of ordinary skill in the art are familiar with formulation and administration techniques that can be employed in use of the invention, e.g., as discussed in Goodman and Gilman's The Pharmacological Basis of Therapeutics, current edition; Pergamon Press; and Remington's Pharmaceutical Sciences (current edition.) Mack Publishing Co., Easton, Pa.

The compounds utilized in the methods of the instant invention may be administered either alone or in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

The pharmaceutical compositions used in the methods of the instant invention can contain the active ingredient in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions used in the methods of the instant invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including mono- or diglycerides. In addition, fatty acids such as oleic acid find use in the preparation of injectables.

The HSP90 inhibitors used in the methods of the present invention may also be administered in the form of a suppositories for rectal administration of the drug. These compositions can be prepared by mixing the inhibitors with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing an HSP90 inhibitor can be used. (As used herein, topical application can include mouth washes and gargles.)

The compounds used in the methods of the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The HSP90 inhibitors used in the instant invention may also be co-administered with other well known therapeutic agents that are selected for their particular usefulness against the condition that is being treated. For example, the instant compounds may be useful in combination with known anti-cancer and cytotoxic agents. The instant compounds may also be useful in combination with other inhibitors of parts of the signaling pathway that links cell surface growth factor receptors to nuclear signals initiating cellular proliferation.

The methods of the present invention may also be useful with other agents that inhibit angiogenesis and thereby inhibit the growth and invasiveness of tumor cells, including, but not limited to VEGF receptor inhibitors, angiostatin and endostatin.

When a HSP90 inhibitor used in the methods of the present invention is administered into a human subject, the daily dosage will normally be determined by the prescribing physician with the dosage generally varying according to the age, weight, and response of the individual patient, as well as the severity of the patient's symptoms.

In one exemplary application, a suitable amount of a HSP90 inhibitor is administered to a mammal undergoing treatment for cancer. Administration occurs in an amount of each type of inhibitor of between about 0.1 mg/kg of body weight to about 60 mg/kg of body weight per day, preferably of between 0.5 mg/kg of body weight to about 40 mg/kg of body weight per day. A particular therapeutic dosage that comprises the instant composition includes from about 0.01 mg to about 1000 mg of a HSP90 inhibitor. Preferably, the dosage comprises from about 1 mg to about 1000 mg of a HSP90 inhibitor.

Examples of antineoplastic agents which can be used in combination with the methods of the present invention include, in general, alkylating agents, anti-metabolites; epidophyllotoxin; an antineoplastic enzyme; a topoisomerase inhibitor; procarbazine; mitoxantrone; platinum coordination complexes; biological response modifiers and growth inhibitors; hormonal/anti-hormonal therapeutic agents and haematopoietic growth factors.

Exemplary classes of antineoplastic agents further include the anthracycline family of drugs, the vinca drugs, the mitomycins, the bleomycins, the cytotoxic nucleosides, the epothilones, discodermolide, the pteridine family of drugs, diynenes and the podophyllotoxins. Particularly useful members of those classes include, for example, caminomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin or podo-phyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

Preferably, the pharmaceutical preparation is in unit dosage form. In such form, the preparation is subdivided into unit doses containing appropriate quantities of the active component, e.g., an effective amount to achieve the desired purpose.

The quantity of active compound in a unit dose of preparation may be varied or adjusted from about 0.1 mg to 1000 mg, preferably from about 1 mg to 300 mg, more preferably 10 mg to 200 mg, according to the particular application.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The amount and frequency of administration of the HSP90 inhibitors used in the methods of the present invention and, if applicable, other chemotherapeutic agents and/or radiation therapy will be regulated according to the judgment of the attending clinician (physician) considering such factors as age, condition and size of the patient as well as severity of the disease being treated. A dosage regimen of the HSP90 inhibitors can be intravenous administration of from 1 mg to 5 gm/day, more preferably 10 mg to 2000 mg/day, more preferably still 10 to 1000 mg/day, and most preferably 50 to 600 mg/day, in one or more preferably two) doses, to block tumor growth.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the patient, and in view of the observed responses of the disease to the administered therapeutic agents.

Also, in general, the HSP90 inhibitor and the chemotherapeutic agent do not have to be administered in the same pharmaceutical composition, and may, because of different physical and chemical characteristics, have to be administered by different routes. For example, the HSP90 inhibitor may be administered orally to generate and maintain good blood levels, while the chemotherapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of HSP90 inhibitor, and chemotherapeutic agent and/or radiation will depend upon the diagnosis of the attending physicians and their judgment of the condition of the patient and the appropriate treatment protocol.

The HSP90 inhibitor, and chemotherapeutic agent and/or radiation may be administered concurrently (e.g., simultaneously, essentially simultaneously or within the same treatment protocol) or sequentially, depending upon the nature of the proliferative disease, the condition of the patient, and the actual choice of chemotherapeutic agent and/or radiation to be administered in conjunction (i.e., within a single treatment protocol) with the HSP90 inhibitor.

If the HSP90 inhibitor, and the chemotherapeutic agent and/or radiation are not administered simultaneously or essentially simultaneously, then the optimum order of administration of the HSP90 inhibitor, and the chemotherapeutic agent and/or radiation, may be different for different tumors. Thus, in certain situations the HSP90 inhibitor may be administered first followed by the administration of the chemotherapeutic agent and/or radiation; and in other situations the chemotherapeutic agent and/or radiation may be administered first followed by the administration of the HSP90 inhibitor. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the chemotherapeutic agent and/or radiation may be administered first, especially if it is a cytotoxic agent, and then the treatment continued with the administration of the HSP90 inhibitor followed, where determined advantageous, by the administration of the chemotherapeutic agent and/or radiation, and so on until the treatment protocol is complete.

Thus, in accordance with experience and knowledge, the practicing physician can modify each protocol for the administration of a component (therapeutic agent—i.e., HSP90 inhibitor, chemotherapeutic agent or radiation) of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

### EXAMPLES

The following examples are illustrative only, and are not intended to be limiting of the invention.

### Example 1

### Cytotoxic Activity of 17AAG on K562 Versus a Normal Cell Type

Grosveld et al., Mol Cell Biol 6(2):607-16 (1986) showed that the chronic myelocytic cell line K562 produces a chimeric bcr/c-abl transcript, making it a suitable model system to demonstrate the methods of the invention. The cell line is widely available, e.g., from American Type Culture Collection (“ATCC”; Manassas, Va., USA; cat# CCL-243) and can be propogated in a variety of media, e.g., ATCC's Iscove's modified Dulbecco's medium with 4 mM L-glutamine adjusted to contain 1.5 g/L sodium bicarbonate, 90%; fetal bovine serum, 10%; 37 C.

### Experimental

To K562 cells (suspension grown in DMEM media supplemented w/10% Fetal Bovine Serum (FBS) and 1 mM HEPES; subcultured biweekly at 100K cells/ml) in a 96 well plate (0.1 ml medium; 2000 cells per well) were added various concentrations of 17-AAG (CF7) and the effects measured over a period of 3-6 days using an MTS assay protocol similar to that offered by Promega Corp (Madison, Wis., US; cat# G5421).

The MTS assay is a colorimetric assay for determining the number of viable cells in proliferation, cytotoxicity or chemosensitivity assays. The CellTiter 96® AQueous Assay is composed of solutions of tetrazolium compound (3-(4,5-dimethylthiazol-2-yl)-5-(3-carboxymethoxyphenyl)-2-(4-sulfophenyl)-2H-tetrazolium, inner salt; MTS) and an electron coupling reagent (phenazine methosulfate) PMS. MTS is bioreduced by cells into a formazan that is soluble in tissue culture medium. Barltrop et al. (1991) Bioorg. & Med. Chem. Lett. 1, 611. The absorbance of the formazan at 490 nm can be measured directly from 96 well assay plates without additional processing. Cory et al. (1991) Cancer Commun. 3, 207; Riss, T. L. and Moravec, R. A. (1992) Mol. Biol. Cell 3 (Suppl.), 184a. The conversion of MTS into the aqueous soluble formazan is accomplished by dehydrogenase enzymes found in metabolically active cells. The quantity of formazan product as measured by the amount of 490 nm absorbance is directly proportional to the number of living cells in culture.

Using the MTS assay, cytotoxicity (defined as “growth inhibition” and not necessarily versus renal proximal tubular endothelial cells (normal cells) was determined as shown in the following Tables. “Sem” refers to standard error of the mean, which is calculated as the standard deviation divided by the square root of the sample size; the numbers reflect triplicate replicates. Dilutions of the compounds were prepared in DMSO and straight DMSO was used as a control corresponding to 100% metabolic activity.

Metabolic Activity

| Conc (uM) | K562 | sem1 | RPTEC | sem1 |
|---|---|---|---|---|
| 10.0000 | 7.89 | 0.56 | 20.10 | 2.64 |
| 3.0000 | 8.12 | 1.02 | 22.01 | 2.49 |
| 1.0000 | 9.51 | 0.59 | 34.01 | 0.19 |
| 0.3000 | 14.40 | 1.53 | 58.03 | 5.09 |
| 0.1000 | 44.06 | 2.76 | 86.46 | 1.51 |
| 0.0300 | 80.12 | 2.29 | 86.40 | 5.96 |
| 0.0100 | 85.94 | 0.06 | 91.81 | 8.22 |
| 0.0030 | 83.00 | 2.25 | 92.73 | 4.79 |
| 0.0010 | 83.81 | 0.73 | 92.26 | 2.97 |
| 0.0003 | 88.00 | 0.40 | 98.69 | 7.16 |

[image]

As demonstrated, the fusion protein cancer line K562 is more sensitive to the HSP90 inhibitor than is the normal cell line, RPTEC. It is expected that this will hold true for a variety of tumor cell lines versus a variety of normal cell lines.

In addition to the effects of 17-AAG on K562 versus RPTEC, the effects of a number of other putative HSP90 inhibitors and control compounds were tested side-by-side per the following Table, where “NEC” refers to no effective concentration.

| | RPTEC | K562 |
|---|---|---|
| Compound | IC₅₀ (nM) | IC₅₀ (nM) |
| CF7 | 400 | 70 |
| DMSO | NEC | NEC |
| 208 | 1000 | 50 |
| 237 | 4000 | 100 |
| 483 | 1000 | 70 |
| 481 | 4000 | 400 |

In the table, compound CF7 is the well known 17-AAG and compounds 207, 208, 237, 483, and 481 have the following formulas.

| Compound # | Formula |
|---|---|
| 208 | 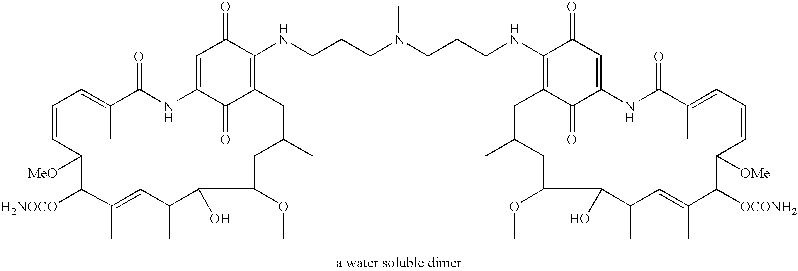 |
| 237 | 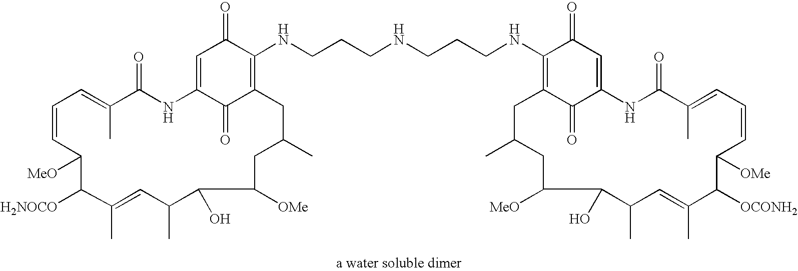 |
| 207 | 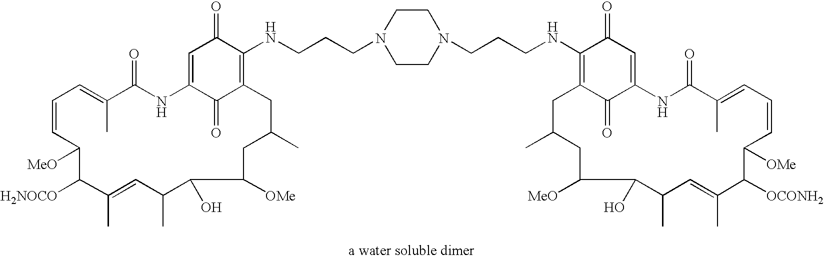 |
| 483 | 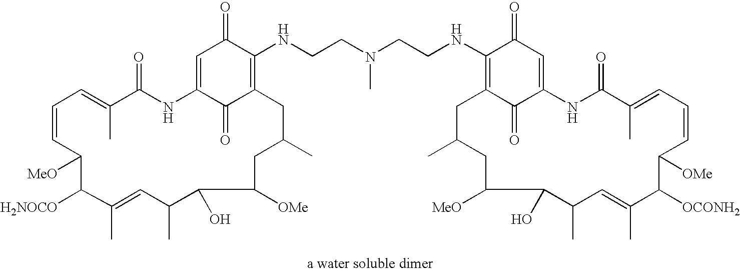 |
| 481 | 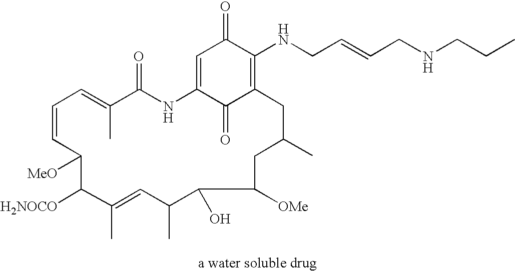 |

A separate study using the well known compound, radicicol, yielded results approximating those obtained for compound 237. Preparation of compounds 207, 208, 237, 483, and 481 is described in the following examples.

### Example 2

### Preparation of Compound #208

3,3′-diamino-N-methyldipropylamine (1.32 g, 9.1 mmol) was added dropwise to a solution of Geldanamycin (10 g, 17.83 mmol) in DMSO (200 ml) in a flame-dried flask under N2 and stirred at room temperature. The reaction mixture was diluted with water after 12 hours. A precipitate was formed and filtered to give the crude product. The crude product was chromatographed by silica chromatography (5% CH3OH/CH2Cl2) to afford the desired dimer as a purple solid (8.92 g, 7.2 mmol). Yield: 81%; mp 153 oC (dec.); 1H NMR (CDCl3) □0.95 (d, J=7 Hz, 6H, 2CH3), 1.0 (d, J=7 Hz, 6H, 2CH3), 1.69 (m, 4H, 2 CH2), 1.74 (m, 4H, 2CH2), 1.76 (s, 6H, 2 CH3), 1.83 (m, 2H, 2CH), 2.0 (s, 6H, 2CH3), 2.3 (s, 3H, N—CH3), 2.36 (dd, J=14 Hz, 2H, 2CH), 2.5 (m, 4H, 2CH2), 2.63 (d, 2H, 2CH), 2.75 (m, 2H, 2CH), 3.25 (s, 6H, 20CH3), 3.35 (s, 6H, 20CH3), 3.4 (m, 2H, 2CH), 3.50 (m, 4H, 2CH2), 3.68 (m, 2H, 2CH), 4.2 (Bs, 2H, OH), 4.3 (d, J=10 Hz, 2H, 2CH), 4.8 (Bs, 4H, 2NH2), 5.19 (s, 2H, 2CH), 5.82 (t, J=15 Hz, 2H, 2CH═), 5.89 (d, J=10 Hz, 2H, 2CH═), 6.59 (t, J=15 Hz, 2H, 2CH═), 6.92 (d, J=10 Hz, 2H, 2CH═), 7.13 (t, 2H, 2NH), 7.24 (s, 2H, 2CH═), 9.21 (s, 2H, 2NH); MS (m/z) 1203 (M+H).

The corresponding HCl salt was prepared by the following method: an HCl solution in EtOH (5 ml, 0.123N) was added to a solution of compound #208 (1 gm as prepared above) in THF (15 ml) and EtOH (50 ml) at room temperature. The reaction mixture was stirred for 10 min. The salt was precipitated, filtered and washed with large amount of EtOH and dried in vacuo.

### Example 3

### Preparation of Compound #207

Compound #207 was prepared by the same method described in example 2 except that 1,4-bis(3-aminopropyl) piperazine was used instead of 3,3′-diamino-N-methyldipropylamine. The pure purple product was obtained after column chromatography (silica gel); yield: 90%; mp 162 oC; ¹H NMR (CDCl3) □0.97 (d, J=6.6 Hz, 6H, 2CH3), 1.0 (d, J=6.6 Hz, 6H, 2CH3), 1.73 (m, 4H, 2 CH2), 1.78 (m, 4H, 2CH2), 1.80 (s, 6H, 2 CH3), 1.85 (m, 2H, 2CH), 2.0 (s, 6H, 2CH3), 2.4 (dd, J=11 Hz, 2H, 2CH), 2.55 (m, 8H, 4CH2), 2.67 (d, J=15 Hz, 2H, 2CH), 2.63 (t, J=10 HZ, 2H, 2CH), 2.78 (t, J=6.5 Hz, 4H, 2CH2), 3.26 (s, 6H, 20CH3), 3.38 (s, 6H, 20CH3), 3.4 (m, 2H, 2CH), 3.60 (m, 4H, 2CH2), 3.75 (m, 2H, 2CH), 4.6 (d, J=10 Hz, 2H, 2CH), 4.65 (Bs, 2H, 20H), 4.8 (Bs, 4H, 2NH2), 5.19 (s, 2H, CH), 5.83 (t, J=15 Hz, 2H, 2CH═), 5.89 (d, J=10 Hz, 2H, 2CH═), 6.58 (t, J=15 Hz, 2H, 2CH═), 6.94 (d, J=10 Hz, 2H, 2CH═), 7.24 (s, 2H, 2CH═), 7.60 (m, 2H, 2NH), 9.20 (s, 2H, 2NH); MS (m/z) 1258 (M+H); The corresponding HCl salt was prepared by the same procedure as described in example 1.

### Example 4

### Preparation of Compound #237

Compound #237 was prepared by the same method described in example 2 except that 3,3′-diamino-dipropylamine was used instead of 3,3′-diamino-N-methyldipropylamine. The pure purple product was obtained after flash chromatography (silica gel); yield: 93%; mp 165° C.; ¹H NMR (CDCl3) □0.97 (d, J=6.6 Hz, 6H, 2CH3), 1.0 (d, J=6.6 Hz, 6H, 2CH3), 1.72 (m, 4H, 2 CH2), 1.78 (m, 4H, 2CH2), 1.80 (s, 6H, 2 CH3), 1.85 (m, 2H, 2CH), 2.0 (s, 6H, 2CH3), 2.4 (dd, J=11 Hz, 2H, 2CH), 2.67 (d, J=15 Hz, 2H, 2CH), 2.63 (t, J 10 HZ, 2H, 2CH), 2.78 (t, J=6.5 Hz, 4H, 2CH2), 3.26 (s, 6H, 20CH3), 3.38 (s, 6H, 20CH3), 3.4 (m, 2H, 2CH), 3.60 (m, 4H, 2CH2), 3.75 (m, 2H, 2CH), 4.6 (d, J=10 Hz, 2H, 2CH), 4.65 (Bs, 2H, 20H), 4.8 (Bs, 4H, 2NH2), 5.19 (s, 2H, 2CH), 5.83 (t, J=15 Hz, 2H, 2CH═), 5.89 (d, J=10 Hz, 2H, 2CH═), 6.58 (t, J=15 Hz, 2H, 2CH═), 6.94 (d, J=10 Hz, 2H, 2CH═), 7.17 (m, 2H, 2NH), 7.24 (s, 2H, 2CH═), 9.20 (s, 2H, 2NH); MS (m/z) 1189 (M+H); The corresponding HCl salt was prepared by the same procedure as described in example 1.

### Example 5

### Preparation of Compound #483

Compound #483 was prepared by the same method described in example 2 except that 2,2′-diamino-N-methyldiethyllamine was used instead of 3,3′-diamino-N-methyldipropylamine. The pure purple product was obtained after flash chromatography; yield: 90%; mp 167-169 oC; 1H NMR (CDCl3) □0.95 (d, J=7 Hz, 6H, 2CH3), 1.00 (d, J=7 Hz, 6H, 2CH3), 1.85 (m, 4H, 2CH2), 1.75 (s, 6H, 2 CH3), 1.80 (m, 2H, 2CH), 2.0 (s, 6H, 2CH3), 2.30 (s, 3H, N—CH3), 2.30 (dd, J=14 Hz, 2H, 2CH), 2.5 (m, 4H, 2CH2), 2.63 (d, 2H, 2CH), 2.75 (m, 2H, 2CH), 3.25 (s, 6H, 20CH3), 3.35 (s, 6H, 20CH3), 3.4 (m, 2H, 2CH), 3.50 (m, 4H, 2CH2), 3.68 (m, 2H, 2CH), 4.2 (Bs, 2H, OH), 4.30 (d, J=10 Hz, 2H, 2CH), 4.8 (Bs, 4H, 2NH2), 5.19 (s, 2H, 2CH), 5.82 (t, J=15 Hz, 2H, 2CH═), 5.90 (d, J=10 Hz, 2H, 2CH═), 6.59 (t, J=15 Hz, 2H, 2CH═), 6.92 (d, J=10 Hz, 2H, 2CH═), 7.13 (t, 2H, 2NH), 7.24 (s, 2H, 2CH═), 9.20 (s, 2H, 2NH); MS (m/z) 1175 (M+H); The corresponding HCl salt was prepared by the same procedure as described in example 1.

### Example 6

### Preparation of Compound #481

To 200 mg (0.357 mmol) of geldanamycin in 8 ml of dry THF in a flame-dried flask was added 91.6 mg (0.714 mmol) of N-propyl-1,4-diamino-2-butene drop-wise under nitrogen. The reaction mixture was stirred at room temperature for 4 h at which time TLC analysis indicated the reaction was complete. The solvent was removed by rotary evaporation and the crude material was chromatographed (5% CH3OH/CH2Cl2 to 15% CH3OH/CH2Cl2) to afford the desired compound as a purple solid (150 mg, 0.228 mmol); yield: 64%; mp 131 oC; 1H NMR (CDCl3) □0.97 (m, 9H, 3CH3), 1.52 (m, 2H, CH2), 1.72 (m, 3H, CH+CH2), 1.80 (s, 3H, CH3), 2.0 (s, 3H, CH3), 2.38 (dd, J=11 Hz, 11, CH), 2.72 (m, 4H, 2CH, CH2), 3.26 (s, 3H, OCH3), 3.38 (s, 3H, OCH3), 3.46 (m, H, CH), 3.6 (m, H, CH), 4.18 (m, 4H, 2CH2), 4.34 (d, J=10 Hz, 1H, CH), 4.8 (Bs, 2H, NH2), 5.19 (s, 1H, CH), 5.88 (m, 4H, 4CH═), 6.38 (m, 1H, NH), 6.61 (t, J=15 Hz, 1H, CH═), 6.94 (d, J=10 Hz, 1H, CH═), 7.30 (s, H, CH═), 9.16 (s, H, NH); MS (m/z) 658 (M+H). The corresponding HCl salt was prepared by the same procedure as described in example 1.

Various patents, publications, and formulations are within the levels of ordinary skill in the art to which the invention pertains. All documents including the sequence listing cited in this disclosure are incorporated by reference to the same extent as if each reference had been incorporated by reference in its entirety individually, although none is admitted to be prior art.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, are encompassed within the spirit of the invention, and are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group, and exclusions of individual members as appropriate.

1. (canceled)

2. The method of claim 94, wherein said compound is an ansamycin.

3. The method of claim 2, wherein said ansamycin is selected from the group consisting of geldanamycin, 17-AAG, herbimycin A, and macbecin.

4. (canceled)

5. The method of claim 94, wherein said compound is a compound that binds into the ATP-binding site of a HSP90.

6. (canceled)

7. The method of claim 94 wherein said identifying comprises using PCR or LCR to identify a nucleic acid encoding said oncogenic fusion protein.

8. (canceled)

9. The method of claim 94 wherein said identifying comprises using a cytochemical technique.

10. The method of claim 9 wherein said cytochemical technique employs nucleic acid hybridization.

11. (canceled)

12. The method of claim 94 wherein said disease is a hematopoietic disorder.

13. The method of claim 12 wherein said hematopoietic disorder is selected from the group consisting of a T or B cell lymphoma, CML, APL, ALL, AML, NHL, and CMML.

14. The method of claim 94 wherein said disease is characterized by a solid tumor.

15. The method of claim 14 wherein said solid tumor is selected from the group consisting of papillary thyroid carcinoma, Ewing's sarcoma, melanoma, liposarcoma, rhabdomyosarcoma, synovial sarcoma.

16-23. (canceled)

24. The method of claim 94 wherein said chromosomal aberration is a translocation, an inversion, or a deletion.

25-26. (canceled)

27. The method of claim 94 wherein said chromosomal aberration is selected from the group consisting of inv14 (q11; q32); t(9; 22)(q34; q11), t(1; 19)(q23; p13.3), t(17; 19)(q22; p13), t(15; 17)(q21-q11-22), t(11; 17)(q23; q21.1), t(4;11)(q21; q23), t(9; 11)(q21; q23), t(11; 19)(q23; p13), t(X; 11)(q13; q23), t(1; 11)(p32; q23), t(6; 11)(q27; q23), t(11; 17)(q23; q21), t(8; 21)(q22; q22), t(3;21)(q26; q22), 5(16; 21)(p11; q22), t(6; 9)(p23; q34), t(4; 16)(q26; p13), inv(2; 2)(p13; p11.2-14), inv(16)(p13q22), t(5;12)(q33; p13), t(2;5)(2p23; q35), t(9:12)(q34:p13), del(12p), t(15;17)(q22;q12), t(11;17)(q23;q12), t(16:16)(p13;q22), inv(16)(p13;q22), t(9;11)(p22;q23), t(1;22)(p13;q13), t(3;3)(q21;q26), inv(3)(q21 q26), t(3;5)(q21;q31), t(3;5)(q25;q34), t(7;11)(p15;p15), t(8;16)(p1;p13), t(9;12)(q34;p13), t(12;22)(p13;q13), del(5q), del(7q), del(20q), t(11q23), t(12;21)(p13;q22), t(5;12)(q31;p13), t(1;12)(q25;p13), t(12;15)(p13;q25), t(1;12)(q21;p13), t(12;21)(q13;p32), and t(5;7)(q33;q11.2)).

28. The method of claim 94 wherein said chromosomal aberration is a t(9; 22)(q34; q11) optionally characterized by and comprising a sequence selected from any one of SEQ ID NOs 15-26 or a homolog, isoform, or allelic variation thereof.

29-31. (canceled)

32. The method of claim 95, wherein said compound is an ansamycin.

33. The method of claim 32, wherein said ansamycin is selected from the group consisting of geldanamycin, 17-AAG, herbimycin A, and macbecin.

34. (canceled)

35. The method of claim 95 wherein said HSP90-inhibiting compound is a compound that binds the ATP-binding site of a HSP90.

36. The method of claim 95 wherein said cancerous cells are leukemic cells.

37. The method of claim 36 wherein said leukemic cells are selected from the group consisting of a T or B cell lymphoma, CML, APL, ALL, AML, NHL, and CMML.

38. The method of claim 95 wherein said treatment is monitored using one or more techniques selected from the group consisting of PCR, antibody staining, and nucleic acid hybridization, and wherein said techniques are selective for the presence of cancerous cells.

39. The method of any of claims claim 95 wherein said genetically-defined proliferative disorder is a solid tumor.

40. The method of claim 39 wherein said solid tumor is selected from the group consisting of papillary thyroid carcinoma, Ewing's sarcoma, melanoma, liposarcoma, rhabdomyosarcoma, and synovial sarcoma.

41-44. (canceled)

45. The method of claim 95 wherein said fusion protein arises from a chromosomal translocation, a chromosomal inversion, or a chromosomal deletion.

46-47. (canceled)

48. The method of claim 95 wherein said fusion protein is generated from a chromosomal aberration selected from the group consisting of inv14 (q11; q32), t(9; 22)(q34; q11), t(1; 19)(q23; p13.3), t(17; 19)(q22; p13), t(15; 17)(q21-q1′-22), t(11; 17)(q23; q21.1), t(4;11)(q21; q23), t(9; 11)(q21; q23), t(11; 19)(q23; p13), t(X; 11)(q13; q23), t(1; 11)(p32; q23), t(6; 11)(q27; q23), t(11; 17)(q23; q21), t(8; 21)(q22; q22), t(3;21)(q26; q22), 5(16; 21)(p11; q22), t(6; 9)(p23; q34), t(4; 16)(q26; p13), inv(2; 2)(p13; p11.2-14), inv(16)(p13q22), t(5;12)(q33; p13), t(2;5)(2p23; q35), t(9:12)(q34:p13), del(12p), t(15;17)(q22;q12), t(11;17)(q23;q12), t(16:16)(p13;q22), inv(16)(p13;q22), t(9;11)(p22;q23), t(1;22)(p13;q13), t(3;3)(q21;q26), inv(3)(q21 q26), t(3;5)(q21;q31), t(3;5)(q25;q34), t(7;11)(p15;p15), t(8; 16)(p11;p13), t(9;12)(q34;p13), t(12;22)(p13;q13), del(5q), del(7q), del(20q), t(11q23), t(12;21)(p13;q22), t(5;12)(q31;p13), t(1;12)(q25;p13), t(12;15)(p13;q25), t(1;12)(q21;p13), t(12;21)(q13;p32), and t(5;7)(q33;q11.2)).

49-93. (canceled)

94. A method of diagnosing a genetically-defined disease characterized by a chromosomal aberration that yields an oncogenic fusion protein, comprising:
providing a cell, tissue, or fluid sample of a patient suspected of having said genetically-defined disease;
identifying one or more characteristics indicative of said disease in or on said cell, tissue, or fluid sample;
administering to said cell, tissue, or fluid sample a pharmaceutically effective amount of an HSP90-inhibiting compound;
determining the effect of said HSP90-inhibiting compound on said cell, tissue, or fluid sample;
comparing the results obtained to the results obtained from a control cell, tissue, or fluid sample;
wherein said HSP90-inhibiting compound has a greater effect on the cell, tissue, or fluid sample from the patient suspected of having compared to the control cell, tissue, or fluid sample.

95. A method of predicting whether a patient with a genetically-defined disease, which is characterized by a chromosomal aberration that yields an oncogenic fusion protein, will be responsive to treatment with an HSP90-inhibiting compound comprising:
providing a cell, tissue, or fluid sample of said patient;
identifying one or more characteristics indicative of said disease in or on said cell, tissue, or fluid sample;
administering to said cell, tissue, or fluid sample a pharmaceutically effective amount of an HSP90-inhibiting compound;
determining the effect of said HSP90-inhibiting compound on said cell, tissue, or fluid sample;
comparing the results obtained to the results obtained from a control cell, tissue, or fluid sample;
wherein said HSP90-inhibiting compound has a greater effect on the cell, tissue, or fluid sample from the patient with said genetically defined disease compared to the control cell, tissue, or fluid sample.
